SEQUENCE LISTING (12 sequences)

SEQ ID NO: 1 - DNA, 684 bp, unidentified
  misc_feature 1..684: note = Tardigrade (WT CAHS D Protein) (DNA)
  source 1..684: mol_type = other DNA
atgtcagggcgtaacgtggagtcccatatggagcgcaacgaaaaagtggtagtgaataactccggtcatgcggacgtgaaaaaacagcaacaacaggttgaacatacggagttcacgcatacagaagtaaaagccccgctcatccacccagcccctccgattatttcgactggcgccgcgggcttagcggaggaaattgtgggccagggttttactgcgtcagcagcgcgtatctcaggtggcactgccgaagtgcatctgcagccgtcagcggcgatgacagaagaagctcgccgtgatcaggaacgttatcggcaggagcaggaaagtattgcgaaacaacaggaacgggaaatggaaaagaagaccgaagcgtaccgcaagacggcggaggcggaagctgaaaaaattcgtaaagaactggaaaaacaacatgcgcgcgatgtcgaattccgtaaagatctgatcgaatccacgatcgatcgtcagaaacgtgaagtggatctggaagcgaaaatggctaaacgcgagttagatcgtgaaggtcagctggctaaagaagccctggaacgctctcggttagccacgaacgtcgaagttaacttcgattcggcagccgggcatacagtcagtggagggaccactgttagcactagcgataagatggaaattaaacgcaactaa

SEQ ID NO: 2 - protein, 227 aa, unidentified
  REGION 1..227: note = Tardigrade (WT CAHS D Protein) (Protein)
MSGRNVESHMERNEKVVVNNSGHADVKKQQQQVEHTEFTHTEVKAPLIHPAPPIISTGAAGLAEEIVGQGFTASAARISGGTAEVHLQPSAAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNVEVNFDSAAGHTVSGGTTVSTSDKMEIKRN

SEQ ID NO: 3 - DNA, 315 bp, synthetic construct
  misc_feature 1..315: note = Modified Tardigrade CAHS Protein (Mutant Linker CAHS Protein, No C Teminus or N Terminus) (DNA)
  source 1..315: mol_type = other DNA
gcagctatgacggaagaggcacgccgtgatcaagagcgctaccgtcaagagcaagaaagtattgccaaacaacaggagcgcgagatggaaaaaaaaactgaggcataccgtaagacggcagaagccgaagcagagaagattcgcaaagagctggagaaacaacatgcccgtgacgtagaatttcgcaaggatttgatcgagtcaaccatcgaccgccagaaacgcgaggtggaccttgaggctaagatggctaaacgtgagcttgatcgcgaaggccaactggcgaaagaggcgctggaacgctctcggttagcc

SEQ ID NO: 4 - protein, 105 aa, synthetic construct
  REGION 1..105: note = Modified Tardigrade CAHS Protein (Mutant Linker CAHS Protein, No C Teminus or N Terminus) (Protein)
AAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLA

SEQ ID NO: 5 - DNA, 999 bp, synthetic construct
  misc_feature 1..999: note = Modified Tardigrade CAHS Protein (Mutant 2x Linker CAHS Protein) (DNA)
  source 1..999: mol_type = other DNA
atgtcagggcgtaacgtggagtcccatatggagcgcaacgaaaaagtggtagtgaataactccggtcatgcggacgtgaaaaaacagcaacaacaggttgaacatacggagttcacgcatacagaagtaaaagccccgctcatccacccagcccctccgattatttcgactggcgccgcgggcttagcggaggaaattgtgggccagggttttactgcgtcagcagcgcgtatctcaggtggcactgccgaagtgcatctgcagccgtcagcggcgatgacagaagaagctcgccgtgatcaggaacgttatcggcaggagcaggaaagtattgcgaaacaacaggaacgggaaatggaaaagaagaccgaagcgtaccgcaagacggcggaggcggaagctgaaaaaattcgtaaagaactggaaaaacaacatgcgcgcgatgtcgaattccgtaaagatctgatcgaatccacgatcgatcgtcagaaacgtgaagtggatctggaagcgaaaatggctaaacgcgagttagatcgtgaaggtcagctggctaaagaagccctggaacgctctcggttagccacgaacatgacggaggaagcgcgtcgcgatcaagaacgttaccgccaagagcaggagtccatcgcaaaacagcaagaacgtgaaatggaaaaaaagacagaggcttaccgtaagacggcagaagccgaggccgaaaaaatccgcaaggaattggaaaagcagcacgcccgtgatgtagagttccgcaaggacttaatcgaatccaccattgatcgccaaaaacgtgaagtagacctggaagccaagatggcgaagcgtgaattggaccgcgaaggccaattagctaaagaggcgctggagcgtagtcgcctggcgacgaacgtcgaagttaacttcgattcggcagccgggcatacagtcagtggagggaccactgttagcactagcgataagatggaaattaaacgcaactaa

SEQ ID NO: 6 - protein, 336 aa, synthetic construct
  REGION 1..336: note = Modified Tardigrade CAHS Protein (Mutant 2x Linker CAHS Protein) (Protein)
MGSSMSGRNVESHMERNEKVVVNNSGHADVKKQQQQVEHTEFTHTEVKAPLIHPAPPIISTGAAGLAEEIVGQGFTASAARISGGTAEVHLQPSAAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNVEVNFDSAAGHTVSGGTTVSTSDKMEIKRN

SEQ ID NO: 7 - DNA, 684 bp, synthetic construct
  misc_feature 1..684: note = Modified Tardigrade CAHS Protein (Mutant FL-Proline CAHS Protein, Prolines Inserted) (DNA)
  source 1..684: mol_type = other DNA
atgtcgggccgtaacgtcgaatctcacatggaacgtaatgagaaggtggtggtgaacaacagcgggcacgctgatgtcaaaaagcaacagcagcaggtagaacatacggagtttacccatactgaagtgaaagccccattaatccaccctgccccgccgattattagcacgggtgctgccggtttagcggaggaaattgtgggacagggttttacagcgagcgcagctcgtatcagtggaggtacggctgaagtccatctgcaaccaagcgcggcaatgacagaagaagcccgccgtgatcaggagcgctatcgtcaagaacaggagtctattgcaaagcagcaagagcgcgagatggaaaaaaagaccgaagcctaccgcaaaaccgccgaagcagaagccgaaaagattcgtaaggagctggaaaaacaacatgcccgtgatgtagaattccgtaaagatctgatcgagagcaccattgatcgtcaaaaacgcgaggttgatctggaagcaaagatggccaaacgtgaactggatcgtgaggggcagcttgcaaaggaggcactggaacgcagccgcttagccacgaacgtaccagtcaatttcccgagtgccgctggacacacggtttcaggaggaaccaccccatcgaccagtgacaagatgccgatcaaacgtaactaa

SEQ ID NO: 8 - protein, 231 aa, synthetic construct
  REGION 1..231: note = Modified Tardigrade CAHS Protein (Mutant FL-Proline CAHS Protein, Prolines Inserted) (Protein)
MGSSMSGRNVESHMERNEKVVVNNSGHADVKKQQQQVEHTEFTHTEVKAPLIHPAPPIISTGAAGLAEEIVGQGFTASAARISGGTAEVHLQPSAAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNVPVNFPSAAGHTVSGGTTPSTSDKMPIKRN

SEQ ID NO: 9 - DNA, 715 bp, unidentified
  misc_feature 1..715: note = AavLEA (WT) (DNA)
  source 1..715: mol_type = other DNA
cactaccgcttacaaccaatcttcaaacgaaatgtcctctcagcagaaccagaaccgacagggtgagcagcaggagcagggctacatggaggcggccaaggagaaggtcgtcaacgcatgggagagcacgaaggaaaccctctcgagcacggctcaagcggccgccgagaagacggctgagtttcgcgattccgccggtgagaccatccggtaagcaacgattccaagtctatgtctgaaacgtacctatattggcggtgctttctaactctgcgatttagtgacctgaccggacaggcgcaggagaagggtcaggagttcaaggagcgcgctggcgagaaggcagaggagacgaagcagcgtgccggggagaagatggatgagaccaagcagcgggctggcgaaatgcgcgagaacgcgggccagaagatggaggagtacaagcagcagggcaagggcaaggccgaggagcttcgcgacactgccggtaggttttctgttcctattgcattgggcatacgaagtagatttgtccgcctgtaatgcactcaactgaactgtttcagccgagaagctccaccaggctggcgagaaggtcaagggccgcgactaagcggttgccccgtcatcacaccgaccttgttccaccttcatgactttcgttctccgtcgtcataaatattttgttttcgacatctgtcagtgatattcctgt

SEQ ID NO: 10 - protein, 143 aa, unidentified
  REGION 1..143: note = AavLEA (WT) (DNA)
MSSQQNQNRQGEQQEQGYMEAAKEKVVNAWESTKETLSSTAQAAAEKTAEFRDSAGETIRDLTGQAQEKGQEFKERAGEKAEETKQRAGEKMDETKQRAGEMRENAGQKMEEYKQQGKGKAEELRDTAAEKLHQAGEKVKGRD

SEQ ID NO: 11 - DNA, 252 bp, unidentified
  source 1..252: mol_type = other DNA
atgtcaggaccgaatggagacttaggaatgccagtcgaggcgggagcagagggcgaggaagacggatttggggaagctgagtacgcggctataaactcaatgcttgatcaaataaactcttgccttgaccatcttgaggaaaagaatgaccaccttcacgcacggctgcaggagcttctggagagcaaccgtcagacccgtttagagttccaacaacaactgggagaagcgcccagtgacgcgagcccatga

SEQ ID NO: 12 - protein, 83 aa, unidentified
MSGPNGDLGMPVEAGAEGEEDGFGEAEYAAINSMLDQINSCLDHLEEKNDHLHARLQELLESNRQTRLEFQQQLGEAPSDASP

(19) United States
(12) Patent Application Publication
(10) Pub. No.: US 2026/0327276 A1
(43) Pub. Date: Oct. 22, 2026

(54) STABILIZATION OF BIOLOGICS AND HUMAN BLOOD CLOTTING FACTOR VIII IN A DRY STATE

(71) Applicant: UNIVERSITY OF WYOMING, Laramie, WY (US)

(72) Inventors: Silvia SANCHEZ-MARTINEZ, Laramie, WY (US); Thomas C. BOOTHBY, Laramie, WY (US)

(21) Appl. No.: 19/128,455

(22) PCT Filed: Nov. 8, 2023
(86) PCT No.: PCT/US2023/079050

§ 371 Date: May 8, 2025

Related U.S. Application Data
(60) Provisional application No. 63/423,694, filed on Nov. 8, 2022.

(51) Int. Cl. C07K 14/435; A61K 38/37; A61K 47/26; A61K 47/42; C07K 14/47

(52) U.S. Cl. CPC C07K 14/43504; A61K 38/37; A61K 47/26; A61K 47/42; C07K 14/4354; C07K 14/47

(57) ABSTRACT

Embodiments of the present disclosure generally relate to the stabilization of biologics, such as blood clotting factors, in a dry state. Methods and compositions for stabilizing biologics are described and can include an intrinsically disordered protein that can be modified to prevent, or at least mitigate, polymerization thereof and the formation of gel-like matrices, thereby, for example, improving the ability of the intrinsically disordered proteins to protect and stabilize sensitive biologics. In an embodiment is provided a composition that includes an intrinsically disordered protein and a blood clotting factor protein. The composition is characterized as stabilizing the biologic of the composition in a dry state at a temperature of ambient temperature or higher.

### FIELD

Embodiments of the present disclosure generally relate to the stabilization of biologics, such as blood clotting factors, in a dry state.

### REFERENCE TO SEQUENCE LISTING

This application contains references to amino acid and nucleic acid sequences which have been submitted as the sequence listing text file entitled “SEQ ID NOS 1-12”, file size 24 Kilobytes (KB), created Oct. 11, 2023, which is hereby incorporated by reference into this application in its entirety.

### BACKGROUND

Having access to safe and effective medication is important for extending the right of equitable healthcare globally. Medicinal accessibility drives the world's ability to treat, control, and prevent sickness and disease. Unfortunately, many medicines have serious limitations that inhibit their accessibility and reliability in many parts of the world. For example, it is often difficult to transport and/or store pharmaceuticals in regions of the world that do not have dependable medicinal infrastructure. Many therapeutics require freezer storage or have short shelf lives that require specific temperatures or consistent pharmaceutical restocking. While vitally important, drugs that require the aforementioned energy intensive environments are not compatible with many medically underdeveloped communities across the globe. Biologics, drugs made or derived from living organisms, are among the most unstable medicines. Human blood clotting factors are a group of biologic medicines that are essential to have readily available in any and every hospital because of their fast action hemorrhage stabilization abilities. However, blood clotting factors require, for example, cold storage temperatures, lyophilization, or PEGylation for stability, which are extremely energy intensive and require expensive and specialized equipment.

There is a need for new and improved methods and compositions for stabilizing biologics such as blood clotting factors.

### SUMMARY

Embodiments of the present disclosure generally relate to the stabilization of biologics, such as blood clotting factors, in a dry state. Unlike conventional technologies that utilize cold-chain refrigeration, freezing, and lyophilization to store sensitive blood clotting factors, embodiments described herein can protect or preserve blood clotting factors in a dry state at various desired temperatures such as ambient temperature or temperatures higher than ambient temperature.

In an embodiment, an intrinsically disordered protein is provided. The intrinsically disordered protein includes an amino acid sequence having at least 80% identity to one or more of SEQ ID NOs: 2, 4, 6, 8, 10, 12, or a complement thereof.

In another embodiment, an intrinsically disordered protein is provided. The intrinsically disordered protein includes an amino acid sequence encoded by a nucleotide sequence having at least about 80% identity to one or more of SEQ ID NOs: 1, 3, 5, 7, 9, 11, or a complement thereof.

In another embodiment, an intrinsically disordered protein for stabilizing a blood clotting factor protein is provided. The intrinsically disordered protein includes an amino acid sequence having at least 80% identity to one or more of SEQ ID NOs: 2, 4, 6, 8, 10, 12, or a complement thereof.

In another embodiment, a method for stabilizing a biologic is provided. The method includes introducing a first component comprising an intrinsically disordered protein with a second component comprising a biologic to form a composition, the second component being free of the intrinsically disordered protein, the composition comprising: the intrinsically disordered protein; and the biologic, wherein the composition is characterized as stabilizing the biologic of the composition in a dry state at a temperature of ambient temperature or higher.

In another embodiment, a composition is provided. The composition includes a first component that includes an intrinsically disordered protein. The composition further includes a second component that includes a biologic in a dry state, the composition being characterized as stabilizing the biologic of the composition at a temperature of ambient temperature or higher, the composition further comprising 15 wt % or less of water based on a total wt % of the composition, the total wt % of the composition not to exceed 100 wt %.

In another embodiment, a pharmaceutical composition is provided. The pharmaceutical composition includes a composition described herein. The pharmaceutical composition further includes a pharmaceutically acceptable carrier, excipient, adjuvant, or combinations thereof.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a bar graph showing the percent (%) water content of desiccated human blood clotting factor VIII (FVIII) by cycle according to at least one embodiment of the present disclosure.

FIG. 2 is a bar graph showing exemplary data for the effect of an example sugar (trehalose) at different concentrations on the stabilization of FVIII according to at least one embodiment of the present disclosure.

FIG. 3 is a bar graph showing exemplary data for the effect of an example sugar (sucrose) at different concentrations on the stabilization of FVIII according to at least one embodiment of the present disclosure.

FIG. 4 is a bar graph showing exemplary data for the effect of a cytoplasmic abundant heat soluble D (CAHS D) protein, as an example intrinsically disordered protein (IDP), at different concentrations on the stabilization of FVIII according to at least one embodiment of the present disclosure.

FIG. 5 is a bar graph showing exemplary data for the effect of a mutant CAHS D protein, as an example engineered construct, at different concentrations on the stabilization of FVIII according to at least one embodiment of the present disclosure.

FIG. 6 is a bar graph showing exemplary data for the effect of a mutant CAHS D protein, as an example engineered construct, at different concentrations on the stabilization of FVIII according to at least one embodiment of the present disclosure.

FIG. 7 is a bar graph showing exemplary data for the effect of a mutant CAHS D protein, as an example engineered construct, at different concentrations on the stabilization of FVIII according to at least one embodiment of the present disclosure.

FIG. 8 is a bar graph showing exemplary data for the effect of a Late Embryogenesis Abundant (LEA) protein, as an example IDP, at different concentrations on the stabilization of FVIII according to at least one embodiment of the present disclosure.

FIG. 9 is a bar graph showing exemplary data for the effect of a heat-resistant obscure (Hero) protein, as an example IDP, at different concentrations on the stabilization of FVIII according to at least one embodiment of the present disclosure.

FIG. 10A is a bar graph showing exemplary data for the effect of sucrose on the stabilization of FVIII under thermal stress (95° C. for 48 hours) according to at least one embodiment of the present disclosure.

FIG. 10B is a bar graph showing exemplary data for the effect of trehalose on the stabilization of FVIII under thermal stress (95° C. for 48 hours) according to at least one embodiment of the present disclosure.

FIG. 11A is a bar graph showing exemplary data for the effect of lysozyme, as a negative control, on the stabilization of FVIII under thermal stress (95° C. for 48 hours) according to at least one embodiment of the present disclosure.

FIG. 11B is a bar graph showing exemplary data for the effect of a CAHS D protein, as an example IDP, on the stabilization of FVIII under thermal stress (95° C. for 48 hours) according to at least one embodiment of the present disclosure.

FIG. 11C is a bar graph showing exemplary data for the effect of a mutant CAHS D protein, as an example engineered construct, on the stabilization of FVIII under thermal stress (95° C. for 48 hours) according to at least one embodiment of the present disclosure.

FIG. 11D is a bar graph showing exemplary data for the effect of a mutant CAHS D protein, as an example engineered construct, on the stabilization of FVIII under thermal stress (95° C. for 48 hours) according to at least one embodiment of the present disclosure.

FIG. 12A is a bar graph showing exemplary data for the effect of an AavLEA1 protein, as an example IDP, on the stabilization of FVIII under thermal stress (95° C. for 48 hours) according to at least one embodiment of the present disclosure.

FIG. 12B is a bar graph showing exemplary data for the effect of a Hero9 protein, as an example IDP, on the stabilization of FVIII under thermal stress (95° C. for 48 hours) according to at least one embodiment of the present disclosure.

FIG. 13A shows exemplary data for a mutant CAHS D protein, as an example engineered construct, on the stabilization of FVIII in a 10-week time course study according to at least one embodiment of the present disclosure.

FIG. 13B is a bar graph showing exemplary data for a mutant CAHS D protein, as an example engineered construct, on the stabilization of FVIII in a 10-week time course study according to at least one embodiment of the present disclosure.

### DETAILED DESCRIPTION

Embodiments of the present disclosure generally relate to the stabilization of biologics, such as blood clotting factors, in a dry state. The inventors have discovered methods and compositions for storing blood factors in a dry state at a temperature of ambient temperature or higher without, for example, cold-chain refrigeration, freezing, or lyophilization. In some embodiments, biologic stabilization can be achieved by utilizing intrinsically disordered proteins employed by desiccation tolerant organisms to protect themselves when dried. In some embodiments, biologic stabilization can be achieved by utilizing sugars. In some embodiments, biologic stabilization can be achieved by utilizing a combination of an intrinsically disordered protein and a sugar. The intrinsically disordered proteins can include wild type proteins, modified proteins, or combinations thereof. Embodiments described herein can enable protection and preservation of, for example, blood clotting factors, and can be extended to other biologics.

Biologics, for example, drugs or pharmaceuticals derived from or containing living organisms or components of living organisms, are a cornerstone of modern medicine are a cornerstone of modern medicine. However, nearly all biologics have a major deficiency: they are inherently unstable, requiring, for example, storage under constant cold conditions. The so-called ‘cold-chain’, while effective, represents a serious economic and logistical hurdle for deploying biologics in remote, underdeveloped, or austere settings where access to cold-chain infrastructure ranging from refrigerators and freezers to stable electricity is limited. To address this issue, and in some embodiments, the inventors found compositions and processes that can enable biologics to be kept in a desiccated state under not only ambient temperatures but elevated temperatures. Briefly, and in some embodiments, the inventors found that both protein and sugar-based protectants can stabilize the biologic pharmaceutical Human Blood Clotting Factor VIII under repeated dehydration/rehydration cycles, thermal stress, and long-term dry storage conditions. In addition, embodiments described herein include engineered proteins that have, for example, an altered protection function of the wild type protein change. Such engineered proteins were determined to have different biophysical properties from a protein-based mediator of anhydrobiosis derived from a tardigrade, CAHS D. The inventors also found that changing the ability of CAHS D to form hydrogels can make the protein better or worse at providing protection to Human Blood Clotting Factor VIII under different conditions.

Embodiments described herein demonstrate the effectiveness of tardigrade CAHS proteins and other mediators of desiccation tolerance at preserving the function of a biologic without the use of the cold-chain. In addition, embodiments described herein demonstrate that engineering approaches can change natural products to serve specific protective functions, such as coping with desiccation cycling versus thermal stress. Overall, embodiments of the present disclosure show that, for example, life-saving pharmaceuticals can be stabilized in a dry state and out of the cold chain by utilizing, e.g., natural and/or engineered mediators of desiccation tolerance.

As used herein, a “composition” can include component(s) of the composition, reaction product(s) of two or more components of the composition, a remainder balance of remaining starting component(s), or combinations thereof. Compositions of the present disclosure can be prepared by any suitable mixing process.

A total weight percent (wt %) of the composition is based on the wt % of the biologic, the wt % of the IDP, and the wt % of the additional component. And the total wt % of the composition does not exceed 100 wt %.

For purposes of the present disclosure, and unless the context indicates otherwise, the terms “biologic” and “biologic of interest” are used interchangeably such that reference to one includes reference to the other. For example, reference to “biologic” includes reference to “biologic” and “biologic of interest”.

The use of headings is for purposes of convenience only and does not limit the scope of the present disclosure. Embodiments described herein can be combined with other embodiments.

For purposes of the present disclosure, “ambient temperature” refers to a temperature of about 20° C.

As described above, one major drawback of biologics is their fragility. The breakdown, aggregation, and/or modification of biologics due to improper storage or transportation is an enormous economic burden. In 2019, about 35% of all vaccines in developing countries had to be discarded due to improper maintenance, leading to multiple countries reporting losses of greater than $1,500,000 for compromised vaccines alone. Perhaps more troubling, not only does the breakdown of biologics lead to reduced efficacy and economic losses, but in many cases loss of biologic integrity can result in degradation products with detrimental, instead of health-inducing, effects. Because of their inherent instability, the economic loss associated with their breakdown, and the risk of harmful effects being induced by breakdown products, reliable, effective, and economical means of stabilization should be used for these life-saving medicines.

The most widespread method of biologic stabilization is cold storage using what is known as the cold-chain, a system of refrigerators and freezers used during the production, transportation, and storage of a biologic to help maintain its viability. Under ideal circumstances, cold stabilization can be effective, however, in remote or developing parts of the world, purchasing and maintaining the necessary infrastructure such as freezers, electrical systems, and backup generators needed for the cold-chain to work seamlessly can be close to impossible. The cold-chain is currently essential for the stabilization of most biologics. This is because above certain temperatures (for example, about 8° C. for most vaccines), molecular dynamics and rearrangements are accelerated leading to hastened breakdown of these materials. At too cold of temperatures (for example, about 2° C. for most vaccines), water begins to freeze, leading to the formation of ice crystals that can irreparably damage sensitive biologics. Therefore, an effective means of stabilization can prevent or reduce molecular motion and can minimize crystallization.

The cold-chain is not a universally reliable, effective, and economical means of stabilizing biologics. Currently, about 75% of all biologic drugs, including essentially all vaccines, about 15% of all small molecules, as well as myriad biological samples and diagnostic tools rely on the cold-chain for stabilization. In remote or developing parts of the world, purchasing and maintaining the necessary infrastructure such as freezers, electrical systems, and backup generators needed for the cold-chain to work seamlessly can be impractical and close to impossible. A prime example of the economic burden of the cold-chain is the fact that in developing countries between 40 and 90% of the cost of a vaccination program comes from the need to keep vaccines cold. This means that for poorer countries, where it is estimated that about 50% of all healthcare facilities completely lack any electrical infrastructure with only about 10% having access to reliable electricity, the cold-chain is inefficient, unreliable, and not economically viable.

Attempts to improve, simplify, and adapt the cold-chain to accommodate logistical and economic restrictions in developing countries have been made. Notably, two Gates Foundation sponsored projects developed a refrigerator and cooler, both of which can maintain cold temperatures required to stabilize vaccines for at least 5 days without electricity. While these innovations have helped with local distribution in the field, they are still limited (power outage in many rural Africa communities can last weeks) and do not address the fact that the cold-chain must be maintained during production and global distribution (a vaccine must travel over 5,000 miles from the USA to sub-Saharan Africa).

To address this major obstacle, the inventors have found that intrinsically disordered proteins (IDPs), such as those possessed by tardigrades, nematodes, and/or plants, among others, can be used to stabilize biologics under conditions outside of conventional storage methods and/or use of conventional agents such as non-reducing sugars. That is, a biologic can be stabilized via the use of intrinsically disordered proteins and stored at temperatures or conditions outside of the biologic's normal or conventional storage temperatures and conditions. For example, and in some embodiments, the compositions described herein can enable, for example, stabilization of a biologic under extreme drought conditions, desiccating conditions, or near-complete water loss, and/or up to about 100° C. via the use of intrinsically disordered proteins. When the biologic is ready to be used, an additive such as a fluid (such as water or buffer solution), a gel, a solid, another additive, or combinations of additives can be added to the biologic.

Tardigrades, also known as water bears, make up a phylum of small but extremely hardy animals and have the ability to survive extreme stresses, including desiccation. These desiccation-tolerant organisms possess several stress-tolerant intrinsically disordered proteins such as cytoplasmic abundant heat soluble proteins, also known as cytosolic abundant heat soluble (CAHS) proteins. These CAHS proteins are expressed at high levels as tardigrades desiccate and polymerize to form gels that slow diffusion, preventing desiccation-sensitive proteins from becoming nonfunctional during desiccation and upon rehydration

The inventors have found that the protection and stabilization of a biologic via the use of CAHS proteins can be concentration dependent, with higher concentrations of CAHS proteins providing greater protection and stabilization. However, at high concentrations, CAHS proteins have a propensity to self-associate and form gel-like substances. Due to this propensity of CAHS proteins to form unwanted gels, it is desirable to disrupt or modify CAHS proteins in order to prevent polymerization thereof and the formation of gels. Accordingly, such modifications increase the ability of the CAHS proteins to protect and stabilize sensitive biologics.

Accordingly, and in some embodiments, novel constructs of CAHS proteins having specific mutations and/or modifications that prevent the CAHS proteins from self-assembling and forming gels are described. These modified or mutant CAHS proteins can provide improved ability to protect and stabilize sensitive biologics, especially under extreme conditions such as high temperature, freezing, and/or desiccation. Embodiments of the present disclosure further provide methods for forming these novel constructs, as well as methods for preparing solid or liquid compositions for stabilizing a biologic of interest with these novel constructs.

In addition, the inventors have found that Late Embryogenesis Abundant (LEA) proteins can be effective protectants of a biologic in the dry state. For example, a protective intrinsically disordered protein (IDP) derived from a desiccation tolerant nematode worm LEA (AavLEA1), from Aphelenchus avenae, shows protective effects to biologics across a wide range of concentrations. The inventors have also found that the protection and stabilization of a biologic via the use of LEA proteins can be concentration dependent. In some embodiments, constructs of LEA proteins with specific mutations and/or modifications are used to protect biologics. Embodiments of the present disclosure further provide methods for forming these novel constructs, as well as methods for preparing solid or liquid compositions for stabilizing a biologic of interest with these novel constructs.

The inventors have also found that heat-resistant obscure (Hero) proteins, such as Hero9, can be effective protectants of a biologic in the dry state. Hero9 belongs to a newly discovered class of proteins present in the human proteome.

In some embodiments, an IDP, a sugar, or combinations thereof, among other mediators of desiccation tolerance, are utilized to stabilize a biologic such as Human Blood Clotting Factor VIII (FVIII), in a dry state. The biologic can also be stabilized at temperatures at which they would normally degrade (for example, higher than about 8° C., such as higher than 10° C., higher than 15° C., at ambient temperature or even higher temperatures such as 100° C.). Other biologics are also contemplated. In some examples, CAHS D can provide protection to FVIII at concentrations below its gelation point. In some examples, gel forms of IDPs are utilized to stabilize a biologic. Here, and in some embodiments, the inventors tested whether gelation inhibits protection of FVIII by utilizing three CAHS D variants, one of which gels and two of which do not.

### Example Compositions

Embodiments of the present disclosure generally relate to stabilized biologic compositions, such as stabilized blood clotting factor compositions. Embodiments described herein can be used to stabilize the biologic at temperatures of, for example, ambient temperature or higher and in a dry state.

In some embodiments, the compositions include an intrinsically disordered protein (IDP) and a biologic. As discussed above, most biologics (for example, vaccines, protein and nucleic acid based pharmaceuticals, among others) are conventionally stored under certain conditions (for example, cold-chain) in order to maintain integrity. Even under seemingly ideal storage conditions, the biologics have limited shelf-life, and under non-ideal conditions, the shelf-life of the biologics can be limited to minutes or hours. The fragility of biologics pharmaceuticals presents an enormous economic and logistical burden. The inventors have found that, for example, an intrinsically disordered protein can be utilized to stabilize the biologic under, for example, non-ideal conditions.

The compositions described herein can include at least two components. The first component includes the intrinsically disordered protein (IDP) and the second component includes the biologic. The second component is free of the IDP. Free of the IDP means that the second component (for example, the biologic) is not the IDP. The compositions can be in the form of a solid composition, liquid composition, and/or other compositions as described below.

The IDP can be a naturally occurring protein, a non-naturally occurring protein or a combination thereof. In some examples, the IDP is produced by organisms known as tardigrades. If desired, the IDP can be modified or mutated to, for example, eliminate or at least mitigate an IDP's propensity to polymerize and gel. Such mutated or modified IDPs can be less prone to polymerization and can have an improved ability to protect and stabilize the biologic.

In some embodiments, the IDP includes a wild type IDP, a modified IDP, or combinations thereof. In some examples, the IDP includes a CAHS protein, a modified CAHS protein, a Late Embryogenesis Abundant (LEA) protein, a modified LEA protein, a heat-resistant obscure (Hero) protein, a modified Hero protein, or combinations thereof.

The term “modified” is used interchangeably with the terms “mutant”, “variant”, and “construct” such that reference to one includes reference to the other. When referring to a protein, the modified protein is an engineered construct that is different from the wild type protein.

The second component of compositions described herein includes a biologic. The biologic can be in its natural state, in a modified state, derived from a living organism, and/or synthesized. The biologic can include a peptide, a polypeptide, a protein, an enzyme, an antibody, a globular protein, a hormone, a natural product, a derivative thereof, a component thereof, or combinations thereof, among others. In some embodiments, the biologic includes a drug or a pharmaceutical containing or derived from living organisms such as vaccines, protein-based pharmaceuticals, nucleic acid-based pharmaceuticals, cell-based therapeutics, allergens, anti-venoms, or combinations thereof, among others. The biologic can include an antibody, stem cell, blood, blood product, derivative thereof, a component thereof, or combinations thereof.

The biologic can be heterologous to the IDP. For example, a heterologous polypeptide refers to a non-IDP polypeptide, a non-tardigrade polypeptide, or a polypeptide that is heterologous to the organism, to the genus or to the species from which the particular IDP is derived. A heterologous cell, tissue or organ as used herein, refers to a cell, tissue or organ that is heterologous to the organism, to the genus, or to the species that naturally produces the particular IDP.

As described above, the biologic can be an antibody. The antibody can be any suitable type of immunoglobulin, such as IgA, IgD, IgE, IgG, and IgM. The antibody can be monoclonal or polyclonal and can be of any species of origin, including, for example, camel, goat, human, mouse, rat, rabbit, horse, sheep, or can be a chimeric antibody. The antibody can be a recombinant monoclonal antibody. The antibody can also be chemically constructed. The antibody can also be an antibody fragment, for example, Fab, Fab′, F(ab′)₂, and/or Fv fragments; domain antibodies; diabodies; vaccibodies; linear antibodies; single-chain antibody molecules; and/or multi-specific antibodies formed from antibody fragments. Also included are antibodies which are altered or mutated for compatibility with species other than the species in which the antibody was produced. For example, antibodies can be humanized or camelized. Humanized forms of non-human (for example, murine) antibodies are chimeric immunoglobulins, immunoglobulin chains, or fragments thereof (such as Fab, Fab′, F(ab′)₂, Fv, and/or other antigen-binding subsequences or portions of antibodies) which can contain minimal sequence derived from non-human immunoglobulin.

The biologic can be in a purified form or it can be in a mixture (unpurified or partially purified). For example, the biologic can be obtained from, for example, an organism (animals, bacteria, fungi, nematodes, plants), the cells of an organism (either cultured or isolated), from serum, and/or from in vitro expression systems, that can then be purified, partially purified, or unpurified. In some embodiments, the biologic so produced can then be protected (stabilized) by contact with an IDP immediately without any further isolation or purification, or the biologic can be contacted with the IDP after the biologic is purified or partially purified. Thus, a mixture can include, for example, cell culture, serum, and/or one or more constituents of an organism or cell thereof, and/or an in vitro expression system, and the like.

Some embodiments of the IDP are discussed above. The compositions described herein can include any number or combination of IDPs, for example, IDPs from various tardigrade genera or species. Accordingly, and in some embodiments, the compositions described herein can comprise, consist essentially of, or consist of at least about 1, 2, 3, 4, 5, 6, 7, 8, 9, 10, 11, 12, 13, 14, 15, 16, 17, 18, 19, or 20 or more different IDPs and/or other IDPs. Each of the foregoing numbers can be preceded by the word “about,” “at least about,” “less than about,” or “more than about,” and any of the foregoing numbers can be used singly to describe an open-ended range or in combination to describe a close-ended range. In some embodiments, compositions described herein can include, for example, about 1 to about 25, about 1 to about 20, about 1 to about 15, about 1 to about 10, about 1 to about 5, about 2 to about 10, about 2 to about 5, about 4 to about 10, about 6 to about 10 different IDPs and/or other IDPs. Any of the foregoing numbers can be used singly to describe an open-ended range or in combination to describe a close-ended range. When a composition described herein includes two or more IDPs, the IDPs can be from the same or from any combination of different species or genera. For example, when a composition described herein includes two or more IDPs, the IDPs can be from the same species or genera or from any suitable combination of different species or genera.

Illustrative, but non-limiting, tardigrade genera from which the IDP can be obtained include Macrobiotus spp., Isohypsibius spp., Diphascon spp., Echiniscus spp., Minibiotus spp., Doryphoribius spp., Paramacrobiotus spp., Hypsibius spp., Milnesium spp., Pseudechiniscus spp., Ramazzottius spp., Batillipes spp., Bryodelphax spp., Dactylobiotus spp., Echiniscoides spp., Calcarobiotus spp., Tenuibiotus spp., Itaquascon spp., Cornechiniscus spp., Halechiniscus spp., or combinations thereof. In some embodiments, the IDP is obtained from the tardigrade genera of Hypsibius spp., Paramacrobiotus spp., Milnesium spp. Ramazzottius spp., or combinations thereof. Any suitable number or combination of IDPs from any suitable tardigrade genus or species can be used.

Illustrative, but non-limiting, tardigrade species from which the IDP can be obtained include Hypsibius dujardini, Paramacrobiotus richters, Milnesium tardigradum, Ramazzottius varieornatus, or combinations thereof. Other tardigrade species are contemplated.

The IDP can include a protein or polypeptide (which can be isolated) comprising, consisting essentially of, or consisting of:
(a) an amino acid sequence of any one of SEQ ID NOs: 2, 4, 6, 8, 10, 12, or a complement thereof;
(b) an amino acid sequence encoded by a nucleotide sequence of any one of SEQ ID NOs: 1, 3, 5, 7, 9, 11, or a complement thereof;
(c) an amino acid sequence having at least about 50%, at least about 60%, at least about 70%, at least about 80%, at least about 90%, or at least about 95% identity to one or more of SEQ ID Nos: 2, 4, 6, 8, 10, 12, or a complement thereof,
(d) an amino acid sequence encoded by a nucleotide sequence having at least about 50%, at least about 60%, at least about 70%, at least about 80%, at least about 90%, or at least about 95% identity to one or more of SEQ ID Nos: 1, 3, 5, 7, 9, 11, or a complement thereof, and/or
(e) a functional fragment of any one of (a) to (d).

Polypeptides and fragments thereof can be modified for use by the addition, at the amino- and/or carboxyl-terminal ends, of a blocking agent. Such blocking agents can include, for example, additional related or unrelated peptide sequences that can be attached to the amino and/or carboxyl terminal residues of the peptide to be administered. For example, one or more non-naturally occurring amino acids, such as D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, for example, by acetylation of the N-terminus and/or amidation of the C-terminus. Likewise, the peptides can be covalently or noncovalently coupled to pharmaceutically acceptable “carrier” proteins prior to use.

Additionally provided herein is a recombinant nucleic acid construct comprising, consisting essentially of, or consisting of:
(a) a nucleotide sequence of any one of SEQ ID NOs: 1, 3, 5, 7, 9, 11, or a complement thereof,
(b) a nucleotide sequence that encodes a polypeptide comprising an amino acid sequence of any one of SEQ ID NOs: 2, 4, 6, 8, 10, 12, or a complement thereof;
(c) a nucleotide sequence having at least about 50%, at least about 60%, at least about 70%, at least about 80%, at least about 90%, or at least about 95% identity to a nucleotide sequence of any one of (a) or (b);
(d) a nucleotide sequence which anneals under stringent hybridization conditions to the nucleotide sequence of any one of (a) to (c), or a complement thereof,
(e) a nucleotide sequence that differs from the nucleotide sequences of any one of (a) to (d) above due to the degeneracy of the genetic code;
(f) a functional fragment of a nucleotide sequence of any one of (a) to (e); or
(g) any combination of the nucleotide sequences of (a)-(f).

In some embodiments, the nucleic acid, nucleotide sequence, or polynucleotide described herein can be a complement (which can be either a full complement or a partial complement) of a nucleic acid, nucleotide sequence, or polynucleotide of the present disclosure. Two nucleotide sequences can be considered to be substantially complementary when the two sequences hybridize to each other under stringent conditions. In some embodiments, two nucleotide sequences considered to be substantially complementary hybridize to each other under highly stringent conditions. Stringent conditions refers to a melting temperature above 65° C., indicating the strength of the hybridization.

In some embodiments, the nucleotide sequences and/or recombinant nucleic acid molecules of the present disclosure can be operatively linked and/or associated with a variety of promoters for expression in cells. Thus, in some embodiments, a recombinant nucleic acid described herein can further include one or more promoters operably linked to one or more nucleotide sequences.

The recombinant nucleic acid molecule can be an expression cassette or can be included within an expression cassette. As used herein, “expression cassette” refers to a recombinant nucleic acid molecule comprising a nucleotide sequence of interest (for example, a nucleotide sequence encoding an amino acid sequence having at least about 50%, at least about 60%, at least about 70%, at least about 80%, at least about 90%, or at least about 95% identity to any one of SEQ ID NOs: 2, 4, 6, 8, 10, 12, or a complement thereof; and/or a nucleotide sequence having at least about 50%, at least about 60%, at least about 70%, at least about 80%, about at least 90%, or at least about 95% identity to any one of SEQ ID NOs: 1, 3, 5, 7, 9, 11, or a complement thereof, wherein said nucleotide sequence can be operably associated with at least a control sequence (for example, a promoter). Accordingly, some embodiments of the present disclosure provide expression cassettes designed to express the nucleotide sequences described herein in a cell.

An expression cassette comprising a nucleotide sequence can be chimeric, meaning that at least one of its components is heterologous with respect to at least one of its other components. An expression cassette can also be one that is naturally occurring but has been obtained in a recombinant form useful for heterologous expression. An expression cassette also can optionally include a transcriptional and/or translational termination region (i.e., termination region) that is functional in the cell in which the nucleotide sequence of interest is to be expressed.

A variety of transcriptional terminators are available for use in expression cassettes and are responsible for the termination of transcription beyond the heterologous nucleotide sequence of interest and correct mRNA polyadenylation. The termination region can be native to the transcriptional initiation region, can be native to the operably linked nucleotide sequence of interest, can be native to the host organism, or can be derived from another source (i.e., foreign or heterologous to the promoter, the nucleotide sequence of interest, the host organism, or any combination thereof). In addition, and in some embodiments, a coding sequence's native transcription terminator can be used.

An expression cassette can include a nucleotide sequence for a selectable marker, which can be used to select a transformed organism and/or cell. As used herein, “selectable marker” refers to a nucleotide sequence that when expressed imparts a distinct phenotype to the transformed organism or cell expressing the marker and thus allows such transformed organisms or cells to be distinguished from those that do not have the marker. Such a nucleotide sequence can encode either a selectable or screenable marker, depending on whether the marker confers a trait that can be selected for by chemical means, such as by using a selective agent (for example, an antibiotic, herbicide, or the like), or on whether the marker is simply a trait that one can identify through observation or testing, such as by screening. Of course, many examples of suitable selectable markers useful in various organisms are known in the art and can be used in the expression cassettes described herein.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector includes a nucleic acid molecule comprising the nucleotide sequence(s) to be transferred, delivered, and/or introduced. Vectors for use in transformation of animals, plants, and other organisms are well known in the art. Illustrative, but non-limiting, examples of general classes of vectors include a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector can transform prokaryotic or eukaryotic host either by integration into the cellular genome or exist extrachromosomally (for example, an autonomous replicating plasmid with an origin of replication). Additionally included are shuttle vectors by which is meant a DNA vehicle capable, naturally or by design, of replication in two different host organisms, which can be selected from prokaryotic and eukaryotic organisms. In some embodiments, the nucleic acid in the vector is under the control of, and operably linked to, an appropriate promoter or other regulatory elements for transcription in a host cell such as a microbial, for example, bacterial, or an animal or a plant cell. The vector can be a bi-functional expression vector which functions in multiple hosts. In the case of genomic DNA, this can contain its own promoter or other regulatory elements and in the case of cDNA this can be under the control of an appropriate promoter or other regulatory elements for expression in the host cell.

In some embodiments, the IDP includes one or more wild type IDPs such as a wild type CAHS protein, a wild type Aavlea protein, a wild type Hero protein, or combinations thereof. Additionally, or alternatively, and in some embodiments, the IDP includes a modified version of the wild type IDP, such as a modified CAHS protein, a modified Aavlea protein, a modified Hero protein, or combinations thereof. As described herein, the inventors have found that IDPs can be effective protectants of a biologic in the dry state, outcompeting well known excipients. For example, a CAHS protein, such as CAHS D proteins, is a highly charged 227-residue disordered protein. During its expression and purification, CAHS D proteins undergo a sol-gel phase transition, transitioning from a liquid into a solid gel state. Protection via CAHS D proteins can be concentration dependent, with higher concentrations providing greater protection. However, CAHS D proteins can have an increased propensity to form gels at high concentration. Modifications to CAHS proteins can make such proteins less prone to polymerization and have an improved ability to protect and stabilize the biologic.

Wild type CAHS D proteins have an N-terminus, a C-terminus, and a linker region connecting the two termini. Modified CAHS D proteins (engineered constructs) that can be utilized as at least a portion of the IDP can include:
(a) a CAHS D protein construct having a larger linker region than the wild type protein (for example, a 2× linker as the wild type CAHS D protein or other larger linker regions);
(b) a CAHS D protein construct having a shorter linker region than the wild type protein (for example, 0.5× linker as the wild type CAHS D protein or other shorter linker regions);
(c) a CAHS D protein construct having only a linker region of the wild type protein;
(d) a CAHS D protein construct having one or more prolines inserted into one or more portions of the wild type protein;
(e) a CAHS D protein construct missing at least a portion of the C-terminus of the wild type protein;
(f) a CAHS D protein construct missing at least a portion of the N-terminus of the wild type protein;
(g) a CAHS D protein construct missing at least a portion of the linker region of the wild type protein;
(h) a CAHS D protein construct having only the C-terminus of the wild type protein;
(i) a CAHS D protein construct having only the N-terminus of the wild type protein;
(j) a CAHS D protein construct having two N-termini connected by at least a portion of the linker region of the wild type protein;
(i) a CAHS D protein construct having two C-termini connected by at least a portion of the linker region of the wild type protein;
(j) derivatives thereof, and/or
(k) combinations thereof.

Wild type LEA proteins have an N-terminus, a C-terminus, and a linker region connecting the two termini. Modified LEA proteins (engineered constructs) that can be utilized as at least a portion of the IDP can include:
(a) a LEA protein construct having a larger linker region than the wild type protein (for example, 2× linker as the wild type LEA protein or other larger linker regions);
(b) a LEA protein construct having a shorter linker region than the wild type protein (for example, 0.5× linker as the wild type LEA protein or other shorter linker regions);
(c) a LEA protein construct having only a linker region of the wild type protein;
(d) a LEA protein construct having one or more prolines inserted into one or more portions of the wild type protein;
(e) a LEA protein construct missing at least a portion of the C-terminus of the wild type protein;
(f) a LEA protein construct missing at least a portion of the N-terminus of the wild type protein;
(g) a LEA protein construct missing at least a portion of the linker region of the wild type protein;
(h) a LEA protein construct having only the C-terminus of the wild type protein;
(i) a LEA protein construct having only the N-terminus of the wild type protein;
(j) a LEA protein construct having two N-termini connected by at least a portion of the linker region of the wild type protein;
(i) a LEA protein construct having two C-termini connected by at least a portion of the linker region of the wild type protein;
(j) derivatives thereof; and/or
(k) combinations thereof.

In some embodiments, the IDP includes a gelling IDP, a non-gelling IDP, or combinations thereof combination thereof. The IDP can provide a level of protective capacity to the biologic.

The composition comprising the IDP and the biologic can be in the form of a solid (for example, powder, particles, among others), a liquid (for example, a solution, a suspension, among others), other compositions, or combinations thereof. The compositions can be stored under ideal conditions or non-ideal conditions. For example, when the biologic is intended to be stored at an ideal temperature of 4° C. according to conventional methods, the compositions described herein comprising the IDP and the biologic can be stored at this ideal temperature or a non-ideal temperature (for example, greater than about 4° C. or less than about 4° C.). In some embodiments, when dried (for example, as a solid composition) or when in solution (for example, as a liquid composition), the biologic can be stabilized over a range of temperatures from about −80° C. to about 100° C., though other temperatures are contemplated. In at least one embodiment, when dried (for example, as a solid composition) or when in solution (for example, as a liquid composition), the biologic can be stabilized over a range of temperatures from about −80° C. to about 40° C., though other temperatures are contemplated.

As used herein, a “stabilized biologic” and “stabilizing” a biologic refers to maintaining the structure and/or the function of the biologic under either aqueous conditions or dried conditions, or after being frozen and/or dried and then thawed and/or rehydrated. In some embodiments, the biologic can be stable at a temperature from about −80° C. to about 100° C. once the biologic is introduced or contacted with the IDP. In some embodiments, from about 10% to about 100%, from about 10% to about 95%, from about 10% to about 90%, from about 10 to about 85%, from about 10% to about 80%, from about 10% to about 75%, from about 10% to about 70%, from about 10% to about 60%, from about 10% to about 50%, from about 20% to about 100%, from about 20% to about 95%, from about 20% to about 90%, from about 20% to about 85%, from about 20% to about 80%, from about 20% to about 75%, from about 20% to about 70%, from about 20% to about 60%, from about 20% to about 50%, from about 30% to about 100%, from about 30% to about 95%, from about 30% to about 90%, from about 30 to about 85%, from about 30% to about 80%, from about 30% to about 75%, from about 30% to about 70%, from about 30% to about 60%, from about 30% to about 50%, from about 40% to about 100%, from about 40% to about 95%, from about 40% to about 90%, from about 40 to about 85%, from about 40% to about 80%, from about 40% to about 75%, from about 40% to about 70%, from about 40% to about 60%, from about 40% to about 50%, from about 50% to about 100%, from about 50% to about 95%, from about 50% to about 90%, from about 50 to about 85%, from about 50% to about 80%, from about 50% to about 75%, from about 50% to about 70%, from about 50% to about 60%, from about 60% to about 100%, from about 60% to about 95%, from about 60% to about 90%, from about 60 to about 85%, from about 60% to about 80%, from about 60% to about 75%, from about 60% to about 70%, from about 70% to about 100%, from about 70% to about 95%, from about 70% to about 90%, from about 70 to about 85%, from about 70% to about 80%, from about 70% to about 75%, from about 80% to about 100%, from about 80% to about 95%, from about 80% to about 90%, from about 80 to about 85%, from about 90% to about 100%, from about 90% to about 95%, and the like, of the structure and function of the biologic is maintained. Any of the foregoing numbers can be used singly to describe an open-ended range or in combination to describe a close-ended range.

As an example, an IDP, a sugar, or combinations thereof can be utilized to maintain the clotting ability of FVIII.

In some embodiments, a liquid composition is provided comprising, consisting essentially of, or consisting of: an IDP, a sugar, or combinations thereof; and a biologic. The IDP, sugar, and biologic can each, independently, exist as one or more ions in, for example, solution or suspension.

In some embodiments, a solid composition is provided comprising, consisting essentially of, or consisting of: an IDP, a sugar, or combinations thereof; and abiologic (for example, a biologic, a biologic of interest). The total weight percent (total wt %) of compositions described herein does not exceed 100 wt %. In some embodiments, a solid composition can be produced by drying or partially drying a liquid composition.

In some embodiments, a composition (e.g., a solid composition or a liquid composition) includes water. An amount of water in a solid composition can be from about 0 wt % or more, about 15 wt % or less, or combinations thereof, such as from greater than 0 wt % to about 15 wt %, from about 2 wt % to about 12 wt %, from about 5 wt % to about 15 wt %, from about 5 wt % to about 15 wt %, such as from about 7 wt % to about 12 wt %, such as from about 8 wt % to about 10 wt % based on a total wt % of the composition. Any of the foregoing numbers can be used singly to describe an open-ended range or in combination to describe a close-ended range.

As used herein, the term “dry state” when referring to a composition described herein refers to a composition that has 15 wt % or less water based on the total wt % of the composition. In some examples, a composition in a dry state has from about 0 wt % or more, about 15 wt % or less, or combinations thereof, such as from greater than 0 wt % to about 15 wt %, from about 2 wt % to about 12 wt %, from about 5 wt % to about 15 wt %, from about 6 wt % to about 14 wt %, such as from about 7 wt % to about 12 wt %, such as from about 8 wt % to about 10 wt % based on a total wt % of the composition. Any of the foregoing numbers can be used singly to describe an open-ended range or in combination to describe a close-ended range.

As used herein, “partially drying” refers to drying a composition that it comprises less water than when the drying process began. Thus, for example, “partially drying” can refer to removing about 5% to about 90% of the water that was present in the composition or solution prior to initiating the drying process (for example, about 5%, 10%, 15%, 20%, 25%, 30%, 35%, 40%), 45%, 50%, 55%, 60%, 65%, 70%, 75%, 80%, 85%, or 90% (or any range or value therein), though other amounts of water are contemplated. Thus, and in some embodiments, the amount of water removed when a composition or solution is partially dried can be from about 10% to about 90%, about 20% to about 90%, about 30% to about 90%, about 40% to about 90%, about 50% to about 90%, about 60% to about 90%, about 10% to about 80%, about 20% to about 80%, about 30% to about 80%, about 40% to about 80%, about 50% to about 80%, about 60% to about 80%, about 70% to about 80%), about 10% to about 70%, about 20% to about 70%, about 30% to about 70%, about 40% to about 70%, about 50% to about 70%, about 10% to about 50%, about 20% to about 50%, about 30% to about 50%, about 40% to about 50% (or any range or value therein) of the water that was present in the composition or solution prior to initiating the drying process. A partially dried composition can be dried further such that it contains less water than when the further drying began.

In some embodiments, a solid composition of the present disclosure can include a hydration level of about 0 to about 10 g water per gram of dried protein (for example, up to about 10 g water per gram of dried protein; for example, about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 6, 6.5, 7, 7.5, 8, 8.5, 9, 9.5, 10, and any range or value therein), though other hydration levels are contemplated. In at least one embodiment, a solid composition can include a hydration level of about 0 g to about 1 g water per gram of dried protein.

In some embodiments, an amount of the IDP(s) in a composition described herein can be from about 50 wt % to about 99.9 wt % based on the total wt % of the composition, such as from about 55 wt % to about 95 wt %, such as from about 60 wt % to about 90 wt %, such as from about 65 wt % to about 85 wt %, such as from about 75 wt % to about 85 wt %, or from about 80 wt % to about 99.9 wt %, such as from about 82 wt % to about 98 wt %, such as from about 84 wt % to about 96 wt %, such as from about 86 wt % to about 94 wt %, such as from about 88 wt % to about 92 wt %, though other amounts are contemplated. Any of the foregoing numbers can be used singly to describe an open-ended range or in combination to describe a close-ended range.

In some embodiments, a weight ratio of the biologic(s) (such as a blood factor protein) to the IDP(s) in a composition described herein can be from about 1:100 to about 1:1 (for example, about 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:10, 1:5, 1:1 or any range or value therein), though other weight ratios are contemplated. Any of the foregoing numbers can be used singly to describe an open-ended range or in combination to describe a close-ended range. In some examples, a weight ratio of the biologic(s) to the IDP(s) in a composition described herein can be from about 1:10 to about 1:100, such as from about 1:20 to about 1:90, such as from about 1:30 to about 1:80, such as from about 1:40 to about 1:70, such as from about 1:50 to about 1:60, though other ratios are contemplated. Any of the foregoing numbers can be used singly to describe an open-ended range or in combination to describe a close-ended range.

In some embodiments, compositions described herein further include one or more additional components. Illustrative, but non-limiting, additional components include trehalose, sucrose, maltose, lactose, cellobiose, chitobiose, bovine serum albumin, human serum albumin, mannitol, sorbitol, polysorbate, a salt, a buffer, an antioxidant, a preservative, a colorant, a flavorant, or combinations thereof.

In at least one embodiment, compositions described herein further includes a disaccharide. The disaccharide can be selected from the group consisting of trehalose, sucrose, maltose, lactose, cellobiose, chitobiose, and combinations thereof.

In some embodiments, when a composition described herein includes one or more additional components, an amount of the one or more additional components in the composition can be from about 0.01 wt % to about 99 wt % based on a total wt % of the composition, such as from about 0.01 wt % to about 10 wt %, such as from about 0.1 wt % to about 5 wt %, such as from about 0.5 wt % to about 2 wt %, though other amounts are contemplated. Any of the foregoing numbers can be used singly to describe an open-ended range or in combination to describe a close-ended range. The additional component does not include water.

As a non-limiting example, and in some embodiments, an amount of salt in compositions described herein can be from about 0.01 wt % to about 5 wt % or any value or range therein such as those described above. Any appropriate physiologically compatible salt can be used, for example, sodium chloride (NaCl).

As another example, and in some embodiments, an amount of disaccharide in compositions described herein can be from about 0.01 wt % to about 5 wt % or any value or range therein such as those described above.

A pH of a composition described herein can be from about 5 to about 9, such as from about 5.5 to about 8.5, such as from about 6 to about 8, such as from about 6.5 to about 7.5, such as from about 6.5 to 7, or from about 7 to 7.5, though other values are contemplated. Any of the foregoing numbers can be used singly to describe an open-ended range or in combination to describe a close-ended range.

### Example Methods

Embodiments of the present disclosure also generally relate to methods of forming the compositions described above, methods of stabilizing a biologic (such as a blood clotting factor), and to methods of forming a stabilized biologic. Embodiments described herein can be used to stabilize the least one biologic at temperatures of, for example, ambient temperature or higher.

In at least one embodiment, one or both of these methods can generally include introducing or contacting the first component comprising the IDP with the second component comprising the biologic to form the composition comprising the IDP and the biologic, thereby, for example, stabilizing the biologic. Introducing can be performed under conditions that include suitable temperatures, pressures, and rates of introduction of the IDP with the biologic. The conditions can also include mixing the components of the composition by any suitable mixing process.

In some embodiments, the method further includes removing at least a portion of the liquid content of the composition. The liquid content can include aqueous material. Removing at least a portion of the liquid content can include drying, or at least partially drying, the composition that includes the IDP and the biologic. Any suitable method of drying can be utilized such as, for example, air drying, evaporating, dehydrating, desiccating, vacuum desiccating, vacuum drying, spray drying, freeze drying, spray-freeze drying, lyophilizing, foam drying, or combinations thereof, among other suitable methods.

As described above, the composition can be in the form of, for example, a solid (such as particles and/or powders) and/or a liquid. The at least partially drying can be performed during methods of forming the solid composition and/or liquid composition.

When a liquid composition is desired, and in some embodiments, the methods described herein can include introducing or contacting the first component comprising the IDP with the second component comprising the biologic to form the liquid composition comprising the IDP and the biologic, thereby, for example, stabilizing the biologic. In at least one embodiment, a third component that includes an aqueous material (for example, water, buffer, et cetera), an organic material (for example, an alcohol such as ethanol), an additive, or combinations thereof can be introduced to the first component and/or second component prior to introducing, during introducing the first and second components, and/or after introducing the first and second components. In some examples, the first component and/or second component can already be present in the form of a solution/suspension with a third component. In such cases, for example, addition of a third component can be optional. Drying, or at least partially drying, can be performed if desired.

When a solid or substantially solid composition is desired, and in some embodiments, the methods described herein can generally include forming a liquid composition and then forming a solid composition from the liquid composition. In some embodiments, this method includes introducing (or contacting) the first component comprising the IDP with the second component comprising the biologic to form the liquid composition comprising the IDP and the biologic. Introducing can be performed as described above. The method optionally includes use of a third component as described above. The solid composition (or substantially solid composition) can then be formed by drying, or at least partially drying, the liquid composition according to suitable methods described above.

Embodiments of the present disclosure, as described herein, include methods of forming the compositions comprising the IDP and the biologic. Embodiments also include methods of stabilizing the biologic. Embodiments described herein also include methods of forming a stabilized biologic.

In certain embodiments, methods described herein include introducing or contacting the biologic with the IDP to produce a liquid composition that includes the biologic and the IDP. In certain embodiments, methods described herein can further include at least partially drying the liquid composition to produce a solid composition comprising the biologic and the IDP. By either method, the biologic is stabilized. Any suitable drying method can be utilized such as air drying, evaporating, dehydrating, desiccating, vacuum desiccating, vacuum drying, spray drying, freeze drying, spray-freeze drying, lyophilizing, foam drying, or combinations thereof, among other suitable methods.

### Example Uses

Embodiments described herein also generally relate to uses of the compositions described herein. As described above, the IDPs can impart drought or desiccation resistance/tolerance to a biologic. Once the composition that includes the IDP and the biologic is ready for its intended purpose, the composition can be mixed with another component, for example, a carrier, buffer, excipient, combinations thereof, among others.

In some embodiments, a pharmaceutical composition includes a composition described herein and a pharmaceutically acceptable carrier or excipient. The pharmaceutical composition can be formulated with pharmaceutically acceptable carriers, excipients, or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. In one embodiment, the pharmaceutical compositions can include a combination of multiple (for example, two or more) isolated IDPs of the present disclosure.

For purposes of the present disclosure, a “pharmaceutically acceptable carrier” includes any suitable solvent, dispersion medium, coating, antibacterial agent, antifungal agent, isotonic delaying agent, and absorption delaying agent, and the like that are physiologically compatible. In some embodiments, the carrier can be suitable for intravenous, intramuscular, subcutaneous, parenteral, spinal or epidermal administration (for example, by injection or infusion). Depending on the route of administration, the active compound, e.g., antibody or antigen binding fragment thereof, can be coated in a material to protect the compound from the action of acids and other natural conditions that may inactivate the compound.

In some embodiments, the carrier or excipient for use with the composition disclosed herein can include, but is not limited to, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, surfactant polyoxyethylene-sorbitan monooleate, or combinations thereof.

In some examples, a pharmaceutical composition (or a portion thereof) can be prepared by contacting the biologic with the IDP, which is then dried to form a powder composition. When the pharmaceutical composition is ready to be, for example, administered to a patient or animal, the composition in the form of a powder can be reconstituted in, for example, a buffer, and then administered to the patient or animal. Any other biologic described herein can be formulated with the IDP and reconstituted in the same or similar manner.

In yet another embodiment, the present disclosure provides a pharmaceutical pack or kit comprising one or more containers filled with one or more of the ingredients of the pharmaceutical composition described herein, such as the IDPs described herein. Optionally, associated with such container(s) can be a notice in the form prescribed by a governmental agency regulating the manufacture, use or sale of pharmaceuticals or biological products, which notice reflects approval by the agency of manufacture, use or sale for human administration.

In some embodiments, a kit can include a composition described herein (for example, an IDP and a vaccine, antibody, et cetera). In some embodiments, the kit includes one or more additional components, such as carriers, buffers, therapeutic agents, diagnostic agents, food agents, or other components suitable for the intended use. In a specific but non-limiting embodiment, the kit comprises a first container containing a composition described herein. In a specific but non-limiting embodiment, the kit comprises a first container that is a vial containing a composition described herein as a lyophilized sterile powder under vacuum. Kits described herein can further include a second container containing a pharmaceutically acceptable fluid.

The following examples are put forth so as to provide those of ordinary skill in the art with a complete disclosure and description of how to make and use aspects of the present disclosure, and are not intended to limit the scope of embodiments of the present disclosure. Efforts have been made to ensure accuracy with respect to numbers used (for example, amounts, dimensions, etc.) but some experimental errors and deviations should be accounted for.

### Examples

In some examples, wild type CAHS D proteins, mutant constructs of CAHS D proteins, AavLEA1 protein, and Hero9 were investigated for their protective effects on an example protein (human blood clotting factor VIII).

### 1. Materials and Methods

### Example Cloning and Expression of CAHS D, CAHS D Constructs, AavLEA1, and Hero9

Cloning and Expression of CAHS D, CAHS D constructs (also referred to as variants), AavLEA1, and Hero9 were cloned into a pET28b expression vector using Gibson Assembly methods. Expression constructs were transformed in BL21 (DE3) E. coli (New England Biolabs) and plated on Luri-Bertani (LB) agar plates with 50 μg/mL Kanamycin (Kan).

Day 0: O/N starter culture. For 6 Liters, a CAHS D overnight culture by inoculating 100 mL lysogeny broth/Kanamycin (LB/Kan) cultures with one colony of BL21-DE3//pET28b_CAHSD in a sterile 250 mL baffled flask. 6 L of LB for expression was made and autoclaved.

Day 1: Protein Expression. Protein expression was performed as follows:
(1) Kan was added to each flask of LB immediately before use, and swirled to mix the antibiotic completely.
(2) Each liter of LB/Kan was inoculated with 10 mL of saturated overnight culture, shaken at 180 rpm at 37° C. until the culture reached an optical density (OD) of 0.6-0.8 (this usually happens within 2.5 hours, if the overnight has not overgrown but is still saturated).
(3) The OD of the expression cultures were checked periodically, and once the OD reached 0.6 (and has not passed 0.8), expression was induced in each liter with 1 mL of 1 M IPTG (final [IPTG]=1 mM), and expression was allowed to go for 4 hours post induction.
(4) Once expression had gone for 3-4 hours, cells were harvested by centrifugation at 3500×g for 25 minutes to 30 minutes. The supernatant (LB) was poured off, and the pellets shaken to remove residual LB.
(5) 5 mL of pellet resuspension buffer (20 mM Tris; pH of 7.5)+30 μL of 1× protease inhibitor stock was added to each pellet of 1 L expression immediately after removing the LB. Once all pellets had resuspension buffer on them, swirling was performed to resuspend the pellets to ensure complete resuspension. To aid in resuspension, a serological pipette can be used to pipette up/down. Pellets were stored at −80° C.

Day 2: Anion Exchange Purification on a 20 mL SP HP column. Anion exchange was performed as follows (Note: Each AKTA run can purify up to 6 pellets worth of protein, if the column is clean):

### Prepping the Protein Load:

(1) Water was heated until boiling. If the pellets were frozen, the pellets are thawed in a water bath (temperature of 37° C.).
(2) Once the water was boiling and the pellets had thawed, the beaker of boiling water was removed from the heat source and pellet tubes were placed in the hot water, with the caps slightly loosened. The pellet tubes were allowed to sit in the hot water for 15 minutes.
(3) Removed from water and allowed to cool for 5 to 10 minutes.
(4) Spun down at 10,500 rpm for 30 minutes. (Rotor JA-20 at 10,500 rpm). (5) Supernatant was decanted into a clean 50 mL conical.
(6) The supernatant was sterile filtered through a 0.22 um syringe filter/bottle top.
(7) The sterile-filtered supernatant was mixed with double volume of room-temperature UA (for example, 10 mL of supernatant+20 mL of Buffer UA=30 mL total). This is the sample load.

### Equilibrating and Running the AKTA Pure:

(1) Prepared the column: Used Pre Wash large column method from the saved methods. Sample, Buffer A, and Buffer B lines were all put in water for the water wash. Once the water wash was done, transferred all the lines to the Buffer and washed.
(2) Sample loaded: The sample line was put into the sample and outlet line to a flow through collection bottle.
(3) Used the saved method for protein of interest.
(4) Ran the method.
(5) Filled the collection tubes.
(6) Ensured that there was enough of both buffers.
(7) Once the sample load was done, placed the sample to Buffer A line for void volume pass.

It can take couple hours for the fractionation and the system will prompt the user to put into water for cleaning. Depending on the mAU bump, fractions are chose and ran on a gel. Pellets were stored at −80° C. until use.

### Example Protein Purification of CAHS D, CAHS D Constructs, and AavLEA1

Pellets were thawed at room temperature and subjected to heat lysis in boiling water for 10 minutes and allowed to cool for 15 minutes. Samples were then centrifuged at 10,500 rpm for 45 minutes at 10° C. The supernatant was filter-sterilized through a 0.22 μm syringe filter (EZFlow Syringe Filter, Cat. 388-3416-OEM) to remove any insoluble particles. The filtrate was diluted two times the volume with buffer UA (8 M Urea, 50 mM sodium acetate, pH 4). This was loaded onto a HiPrep SP HP 16/10 cation exchange column (Cytiva) and purified on an AKTA Pure, controlled using the UNICORN 7 Workstation pure-BP-exp. AavLEA1 was eluted using 0-70% UB gradient (8 M Urea, 50 mM sodium acetate, and 1 M NaCl, pH 4) and fractionated over 15 column volumes. CAHS D, CAHS D 2× Linker and CAHS D Linker Region were eluted using a 0-40% UB gradient and were fractionated over 15 column volumes.

Purified protein fractions were confirmed using SDS-PAGE and selected fractions were pooled for dialysis in 3.5 kDa tubing in 20 mM sodium phosphate buffer pH 7. This was followed by six rounds of dialysis in Milli-Q water (18.2 MΩcm) at 4 h intervals each. Samples were quantified fluorometrically (Invitrogen Qubit 4 Fluorometer, REF Q33226), flash frozen, then lyophilized for 48 h (Labconco FreeZone 6, Cat. 7752021) and stored at −20° C. until further use.

### Example Protein Purification of Hero9

Pellets were thawed at room temperature and subjected to heat lysis in boiling water for 10 minutes and allowed to cool for 15 minutes. The pellets were further lysed by sonication for 5 minutes, using 40% amplitude and 30 seconds on-30 seconds off cycles on ice. All insoluble components were removed via centrifugation at 10,500 rpm at 10° C. for 30 minutes. The supernatant was filtered with 0.45 μm and 0.22 μm syringe filters (EZFlow Syringe Filter, Cat. 388-3416-OEM). The protein was then purified using an anion exchange HiPrep Q HP 16/10 (Cytiva, Cat. 29018183) on the AKTA Pure 25 L (Cytiva, Cat. #29018224), controlled using the UNICORN 7 Workstation pure-BP-exp. Protein was eluted using a gradient of 0-70% B (25 mM Tris-HCl, 1 M NaCl, pH 7.4), over 20 column volumes. Fractions were assessed by SDS-PAGE and pooled for dialysis in 3.5 kDa MWCO dialysis tubing (SpectraPor 3 Dialysis Membrane, Part No. 132724). Protein was dialyzed at 25° C. for 4 h against 25 mM Tris-HCl, 150 mM NaCl, pH 7.4, then transferred to 25 mM Tris-HCl, 50 mM NaCl, pH 7.4 overnight. This was followed by 4 rounds of 4 h each in Milli-Q water (18.2 MΩcm). Dialyzed samples were quantified fluorometrically (Invitrogen Qubit 4 Fluorometer, REF Q33226), aliquoted in the quantity needed for each assay, lyophilized (Labconco FreeZone 6, Cat. 7,752,021) for 48 h, then stored at −20° C. until use.

Table 1 shows the sequence identification numbers for selected intrinsically disordered proteins and nucleotide sequences encoding the intrinsically disordered proteins.

TABLE 1

| | Nucleotide | Amino Acid |
|---|---|---|
| | Sequence | Sequence |
| Example | Number | Number |
| Wild type CAHS D | 1 | 2 |
| CAHS D Linker Region | 3 | 4 |
| CAHS D 2× Linker | 5 | 6 |
| CAHS D Full Length Proline | 7 | 8 |
| Wild type AavLEA1 | 9 | 10 |
| Hero9 | 11 | 12 |

### Example Formulation of Human Blood Clotting Factor VI and Protectants

Human blood clotting factor VIII (FVIII) was hydrated and diluted from 40 mg lyophilized FVIII (Sigma Aldrich cat. H0920000-3EA). Multiple different FVIII vials have been used, all originating from the same lot (lot 6). Lyophilized FVIII deficient plasma (Helena Biosciences cat. 5193) was rehydrated in accordance with Helena Biosciences blood plasma protocol. Multiple FVIII deficient plasma vials were combined and mixed to mitigate potential clotting speed discrepancies within each vial/batch. All FVIII deficient plasma in this study originated from the same lot (lot 2-22-5193).

### Example Human Blood Clotting Factor VIH Clotting Assay

Pacific Hemostasis standardized coagulation APTT-XL protocol was followed, using FVIII as a model clotting factor in this assay. To test the clotting function, 50 μL of FVIII deficient human blood plasma was treated with 5 μL of 5 mg/mL (25 μg) FVIII. After addition of FVIII into the deficient blood plasma, 50 μL aPPT was added to the mixture and incubated in 37° C. for 3 minutes. After incubation, 50 μL of CaCl₂) heated to 42° C. was added. FVIII function was determined by 50% clotting time and quantified via activated Partial Thrombin Time (aPTT). aPTT was measured using a plate reader (Tecan Spark Cyto 600) to record the absorbance at 405 nm.

### Example 6× Desiccation Protocol

10 μL of FVIII (5 mg/mL) was desiccated for 1 hour in a SpeedVac (Savant SpeedVac SC110 Vacuum Concentrator Model SC110-120) and rehydrated with 10 μL molecular grade water. This cycle of desiccation and rehydration was repeated 6 times for each 6× desiccation treated sample. After the final hour of desiccation, the FVIII was rehydrated in 10 μL molecular grade water and 5 μL (25 μg) of treated FVIII was added to FVIII deficient plasma to quantify subsequent clotting activity. To test the potential protective effects of sugar/peptide excipients, 5 μL of 10 mg/mL (50 μg) FVIII was incubated with 5 μL sugar/peptide excipients at varying concentrations for a final FVIII concentration of 5 mg/mL FVIII prior to being subjected to the 6× desiccation protocol. Following 6× desiccation, aPTT was tested on control and experimental samples using the FVIII clotting assay described above.

### Example Water Content Determination Using Thermogravimetric Analysis (TGA)

Dry samples of FVIII were prepared as described above except that samples were removed after 1, 2, 3, 4, 5, and 6 desiccation cycles. Immediately after sample removal from the vacuum desiccator, samples were run on a TA TGA5500 instrument in 100 μL platinum crucibles (TA 952018.906). Crucibles were tared prior to each run and prior to sample loading. TGA analysis was performed by heating samples from 30° C. to 200° C. at a 10° C./minute ramp. Trios software (TA Instruments TRIOS version #5.0.0.44608) was used to perform analysis of the TGA data.

### Example Desiccation and Heat Stress Protocol

To establish a baseline for heat stress, 10 μL of 5 mg/mL (50 μg) FVIII was desiccated for 1 hour in a SpeedVac (Savant SpeedVac SC110 Vacuum Concentrator Model SC110-120) and then placed at 95° C. for 48 hours. After 48 hours of heating, the FVIII was rehydrated in 10 μL molecular grade water. 5 μL (25 μg) of treated FVIII was added to FVIII deficient plasma to quantify subsequent clotting activity. To test the potential protective effects of sugar/peptide excipients, 5 μL of 10 mg/mL (50 μg) FVIII was treated and incubated with 5 μL sugar/peptide excipients at varying concentrations for a final FVIII concentration of 5 mg/mL FVIII prior to being subjected to the desiccation and heating protocol. Following desiccation and heating, control and experimental samples were tested for aPTT using the FVIII clotting assay described above.

### Example Time Course Experiments

Time course experiments for human blood clotting FVIII function was measured over a period of 10 weeks. 10 samples in triplicate (one sample for each week) of 5 μL of 10 mg/mL FVIII with 5 μL of 20 mg/mL CAHS D Linker Region (20 mg/mL) resulting in 50 μg of FVIII and 10 mg/mL CAHS D Linker Region, were desiccated for 1 hour. 10 μL of 5 mg/mL (50 μg) FVIII without any excipient was desiccated for 1 hour. All dried samples were then placed in 1.5 mL Eppendorf tubes sealed with parafilm. The tubes were left on the benchtop at ambient room temperature (about 20° C.). At one-week intervals, a sample would be rehydrated with 10 μL molecular grade water. The 1st sample set of each experiment being measured 1 week after initial desiccation and so on. 5 μL (25 μg) FVIII would be withdrawn from each sample and used to determine aPTT using the FVIII clotting assay described above.

For the hydrated FVIII samples, samples were prepared in triplicate for hydrated FVIII with no protectant and hydrated FVIII supplemented with 10 mg/mL CAHS D Linker Region. Each replicate of hydrated FVIII with no protectant sample contained 5 mg/mL FVIII in molecular grade water. Each replicate of hydrated FVIII with CAHS D Linker Region contained 5 mg/mL FVIII resuspended in molecular grade water with CAHS D Linker Region at a final concentration of 10 mg/mL. All hydrated samples were placed in 1.5 mL Eppendorf tubes sealed with parafilm. The tubes were left on the benchtop at ambient room temperature (about 20° C.). At one-week intervals for 10 weeks, 5 μL (25 μg) FVIII would be withdrawn from each sample and used to determine an aPTT using the FVIII clotting assay described above.

### Statistics

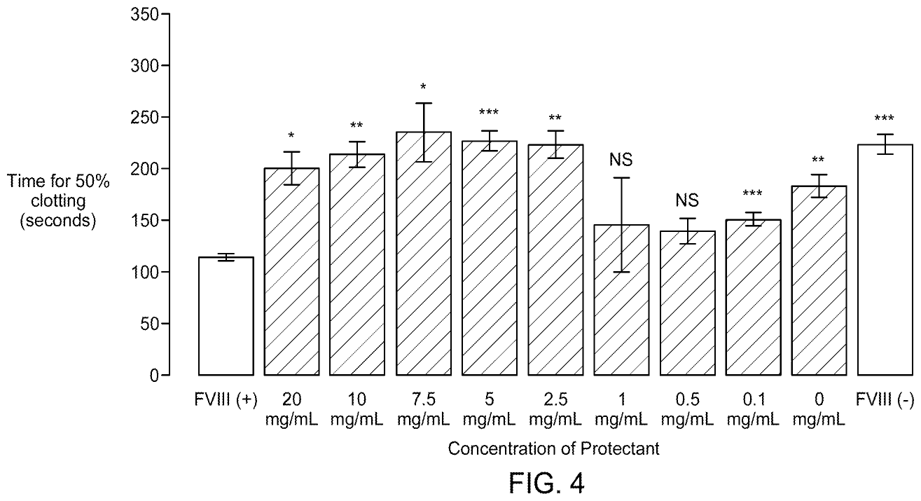

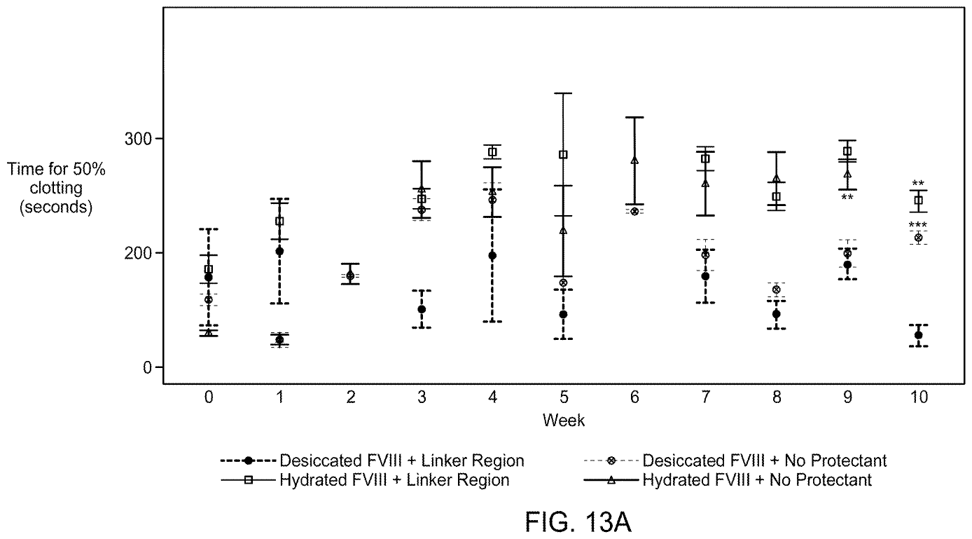

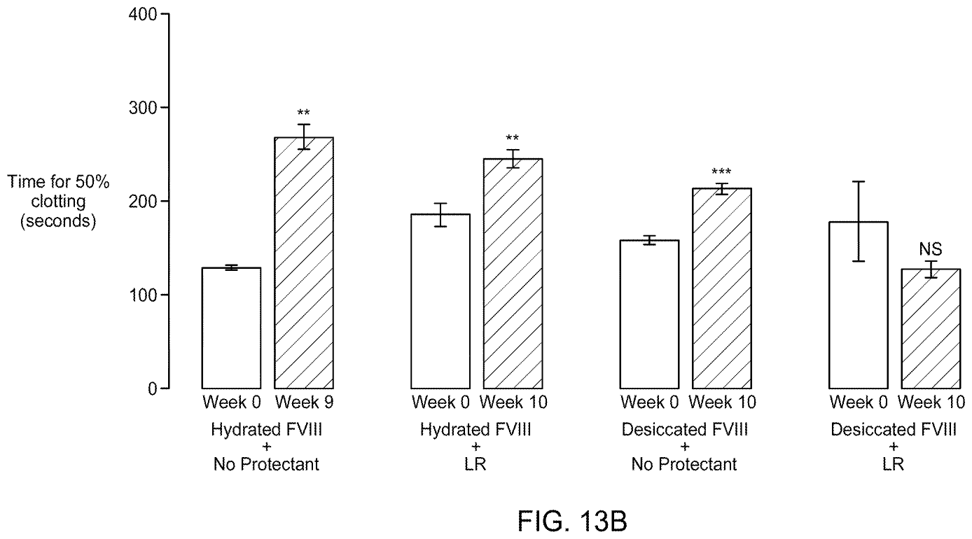

For each clotting experiment, the time for 50% clotting was determined. Using this value, standard deviation of the triplicate within each trial was represented by two sided error bars. In FIGS. 4-9, 10A-10B, 11A-11D, and 12A-12B, significance was determined using one-way Analysis of Variance (ANOVA). For figures using ANOVA, Tukey's post-hoc test was conducted to determine significance values between experimental groups. In FIGS. 13A and 13B, significance values were attained by conducting a two sided T-test comparing clotting speeds from the first and last time point at which clotting was observed.

### 2. Example Stabilization Results

Various, non-limiting, experiments were run to investigate stabilization and protection of blood clotting factors. The inventors found that intrinsically disordered proteins (IDPs), which stabilize biological material of extremotolerant organisms in biologically stressful environments, can protect and preserve, for example, human proteins and enzymes from loss of function/aggregation when desiccated. Extremotolerant organisms, such as the tardigrade, constitutively produce both IDPs and sugars that act to protect an organism's biologic material under desiccation stress. Tardigrade IDPs have been demonstrated to improve desiccation and heat tolerance of desiccation-sensitive cells and proteins in vivo and in vitro. The protective capabilities of tardigrade IDPs have the potential to be utilized to stabilize environmentally sensitive biological pharmaceuticals after desiccation.

The examples show, for example, that IDPs from tardigrades are able to confer significant protection to a biologic pharmaceutical—such as human blood clotting factor VIII-when desiccated and rehydrated. Normally, blood clotting factors require cold storage temperatures, excipients, and lyophilization for stability, which are extremely energy intensive and require expensive and specialized equipment. By using IDP-based stabilizers employed by desiccation tolerant organisms to protect themselves when dried, the inventors show methods and compositions of storing blood factors in a dry state at ambient temperature without the need for cold-chain refrigeration, freezing, or lyophilization. The results also illustrate that, for example, IDPs can be utilized as xeroprotectants to biological medicines.

The examples also show gelation effects on protection. Here, the inventors investigated whether gelation inhibits protection of FVIII by utilizing three CAHS D variants, one of which gels and two of which do not. Under the conditions tested, protection (or lack thereof) can correlate with the gelled state of these variants, with one of the gelling variants (the 2× Linker) protecting FVIII below but not above its gel point. Conversely, and under the conditions tested, two non-gelling variants (named “Full Length Proline” and “Linker Region”) can protect FVIII at a much wider range of concentrations under the conditions tested. The protective effect of AavLEA1, a protective IDP derived from a nematode worm, which does not gel, was also found to provide robust protection to FVIII across a wide range of concentrations.

In the histogram data of FIGS. 4-9, 10A-10B, 11A-11D, and 12A-12B, a protectant at a particular concentration was considered not protective if it was not statistically different from the 0 mg/mL control. A particular protectant concentration is considered partially protective if its 50% clotting time is statistically lower than the 0 mg/mL bar, but also statistically higher than that of plasma supplemented with unperturbed FVIII(+) bar. To be considered fully protective, a protectant concentration's 50% clotting time is statistically lower than the 0 mg/mL bar and statistically indistinguishable from the positive control (FVIII(+) bar).

Faster clotting time is indicated by a lower time (seconds) for 50% clotting.

### Example 2.1: Sugar-Based Mediators for Stabilizing FVIII During Repeated Cycles of Drying and Rehydration

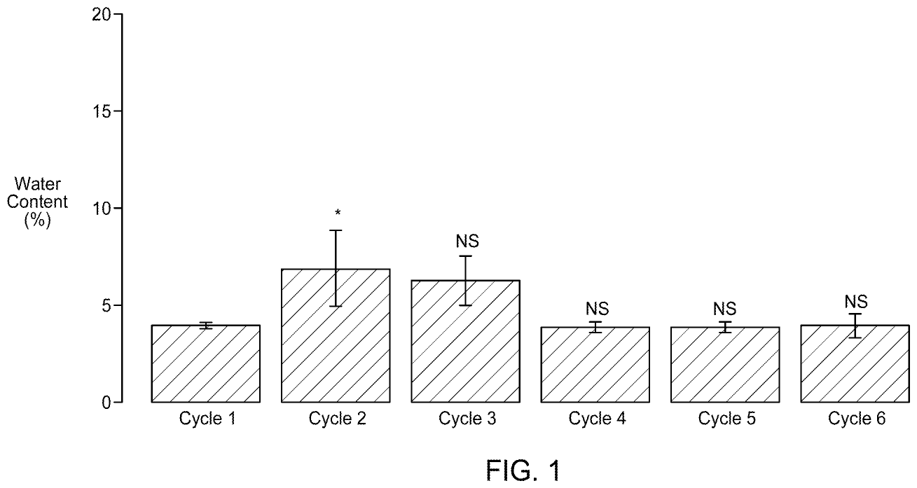

Two non-reducing disaccharides, trehalose and sucrose, were selected as example sugars for initial investigations into whether FVIII could be stabilized and stored in a dry state without refrigeration under non-ideal conditions (e.g., repeated rehydration-dehydration cycles or elevated temperatures). To start, the samples were subjected to 6 repeated cycles of desiccation and rehydration. This stress simulates partial rehydration and dehydration implicit with long-term storage and transit where humidity is not absolutely controlled, such as in clinical settings in remote or developing parts of the world. This desiccation regime also explores the possibility that a dry pharmaceutical could be rehydrated, used, desiccated, and reused in the future. In the 6× desiccation protocol, each sample begins at the same volume and is desiccated under the same drying regime for a selected amount of time, before being rehydrated to its original volume. Each cycle of this protocol uses the same drying and rehydration time, such that the moisture content in desiccated samples closely matched (3.94-6.88%) after each round (FIG. 1). All samples within a single assay were dried simultaneously, thus any minor variation in dryness at the end of each cycle extends to all samples tested and compared within an experiment. FIG. 1 shows the water content of dried FVIII samples after 1, 2, 3, 4, 5, or 6 desiccation/rehydration cycles (n=3). Error bars represent bi-directional standard deviation. Notations above sample bars represent statistical significance determined by one-way ANOVA and Tukey's post-hoc test. P value>0.05=NS, P<0.05=*, P<0.01=**, P<0.001=***.

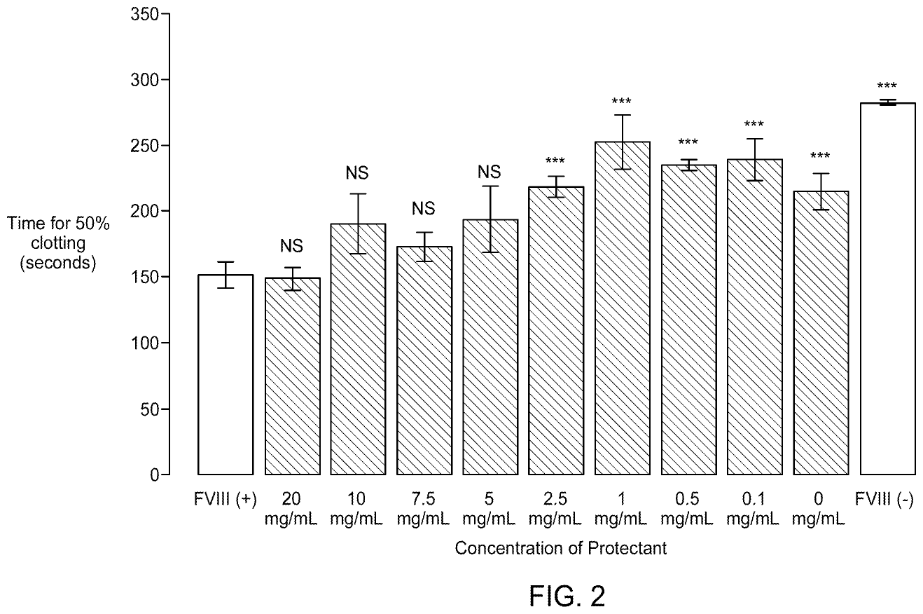

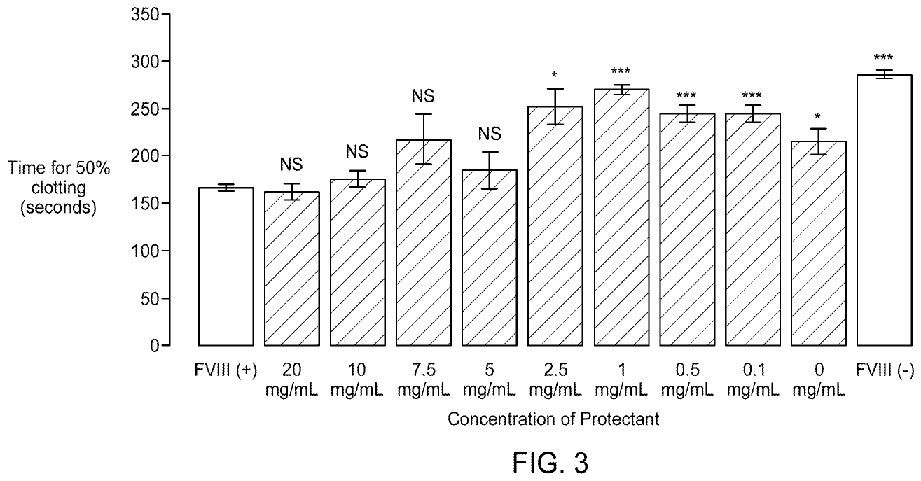

It was determined that trehalose (FIG. 2) and sucrose (FIG. 3) stabilize FVIII under repeated desiccation cycles. In FIGS. 2 and 3, the FVIII(+) bar represents FVIII deficient human blood plasma treated with FVIII that has not been desiccated. Statistical notations above sample bars represent statistical comparison with FVIII(+). “X mg/mL” notations beneath sample bars correspond to the concentrations of non-reducing disaccharide mixed with FVIII before desiccation. The FVIII(−) bar represents FVIII deficient human blood plasma with no supplemented FVIII. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***.

To determine the functionality of FVIII after repeated desiccation and rehydration cycles, FVIII deficient human blood plasma (FVIII DHBP) that, without supplementation of FVIII biologic, clots slowly because it lacks proper intrinsic pathway activation. The functionality of FVIII was evaluated before and after desiccation by mixing it with FVIII DHBP and measuring 50% clotting time. Using this method allows monitoring of the preserved clotting potential of FVIII that had been dried 6 times with or without protectants. This allows the identification of concentration levels of protectants that are not protective, partially protective, or fully protective as described above. To obtain a baseline for healthy blood 50% clotting time, mixed unperturbed FVIII was mixed with FVIII DHBP and it was found that this supplemented plasma had a 50% clotting time of about 150 seconds in the in vitro system (FIGS. 2 and 3, FVIII(+) bars). It is noted that, while single manufacturing lots were used for both FVIII as well as for FVIII DHBP throughout this investigation, individual vials of FVIII and FVIII DHBP vary in their clotting time. As such, each set of experimental replicates performed in this investigation used reagents from the same single vial (or pooled vials when more reagents were needed), so while inter-assay biological variation exists, within a single experiment such variation is not a factor.

To determine a negative control baseline, the 50% clotting time of FVIII DHBP without FVIII addition was measured. FVIII DHBP alone had a 50% clotting time of about 300 seconds (FIGS. 2 and 3, FVIII(−) bars), which is significantly slower than FVIII DHBP treated with healthy FVIII (FIGS. 2 and 3).

To assess the effect of trehalose or sucrose on the 50% clotting time of FVIII DHBP, samples of FVIII DHBP were treated with only sucrose or trehalose, and no FVIII. In these cases, the 50% clotting time sped up to about 270 seconds. While not wishing to be bound by any theory, the fact that both trehalose and sucrose sped up the clotting time may be explained by trehalose inducing a structural transition in aggregation-prone proteins such as alpha-synuclein which leads to accelerated aggregation.

The FVIII was then subjected to 6× desiccation/rehydration stress with either trehalose or sucrose at various concentrations and used these stressed FVIII samples to supplement FVIII DHBP. As shown in FIG. 2, incubating FVIII with increasing concentrations of trehalose prior to desiccation displayed a concentration dependent protective effect, with increasing amounts of trehalose providing increasing levels of protection to desiccated FVIII. At about 5 mg/ml or greater of trehalose, the function of dried FVIII was indistinguishable from that of FVIII(+). Sucrose has a similar chemical structure and protective capacity compared to trehalose. As shown in FIG. 3, the results obtained from sucrose were comparable to trehalose with concentration of about 5 mg/ml or greater displaying protective effects indistinguishable from those of FVIII(+).

As shown in FIGS. 2 and 3, desiccation of FVIII without trehalose or sucrose (0 mg/mL bars) resulted in a clotting factor with compromised clotting ability, indicating that repeated desiccation can perturb FVIII in the absence of excipient protectants. FVIII function was not protected by the addition of 0.1-2.5 mg/mL of sucrose prior to desiccation as shown in FIG. 3. As also shown in FIG. 3, when dried with higher concentrations of sucrose, the function of FVIII was partially protected or fully preserved. A similar concentration dependent trend was observed when trehalose was added to FVIII before desiccation as shown in FIG. 2.

Overall, the data of FIGS. 2 and 3 demonstrates that desiccation without excipients compromises the clotting function of FVIII. Furthermore, although trehalose and sucrose themselves can speed up clotting in FVIII DHBP, it is apparent that they can also protect the function of FVIII during repeated cycles of dehydration and rehydration, because FVIII with 20 mg/mL of sucrose or trehalose caused statistically faster 50% clotting time than FVIII DHBP treated with 20 mg/mL of sucrose or trehalose alone.

### Example 2.2: CAHS D and Engineered CAHS D Variants for Stabilizing FVIII During Repeated Cycles of Drying and Rehydration

To assess whether desiccation-related IDPs can be used to stabilize FVIII, CAHS D was purified and co-incubated with FVIII prior to 6× desiccation cycles. Before testing CAHS D, a baseline was established for how a well-folded protein, unrelated to desiccation tolerance and with no known excipient properties or roles in proteostasis can preserve the function of FVIII during repeated desiccation cycles. To this end, FVIII was treated with a concentration range of lysozyme originating from hen egg-white. Lysozyme is a well characterized and highly structured enzyme with no link to desiccation tolerance. As expected, after 6 desiccation cycles, FVIII treated with lysozyme at any concentration showed at best modest functionality, and at high concentrations lysozyme was antagonistic rather than protective (lysozyme data not shown). This was in contrast to trehalose and sucrose which provide not only partial but also complete protection of FVIII at some concentrations (FIGS. 2 and 3).

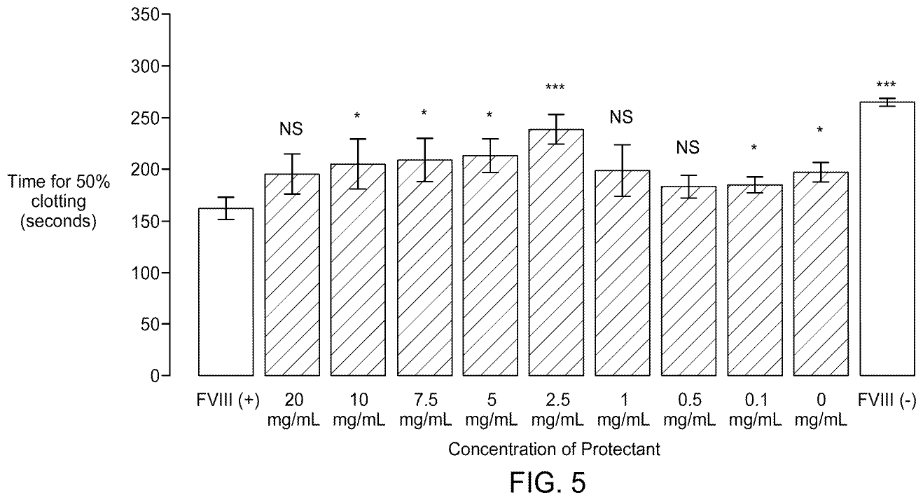

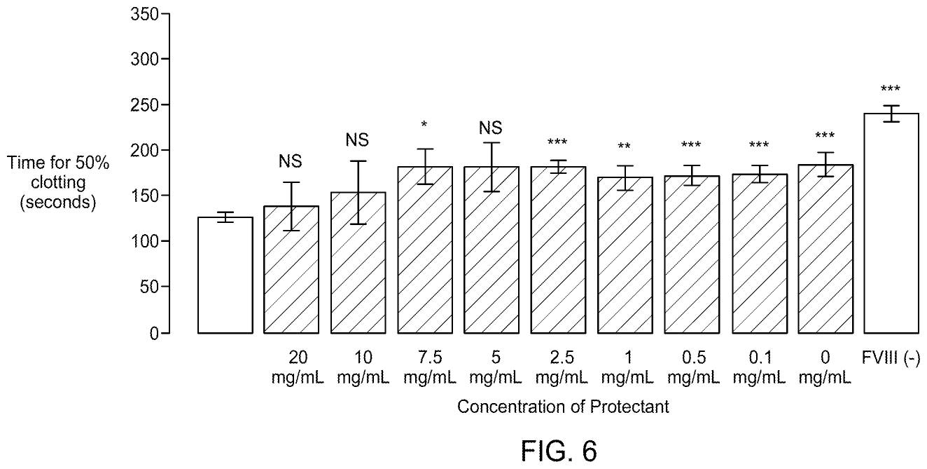

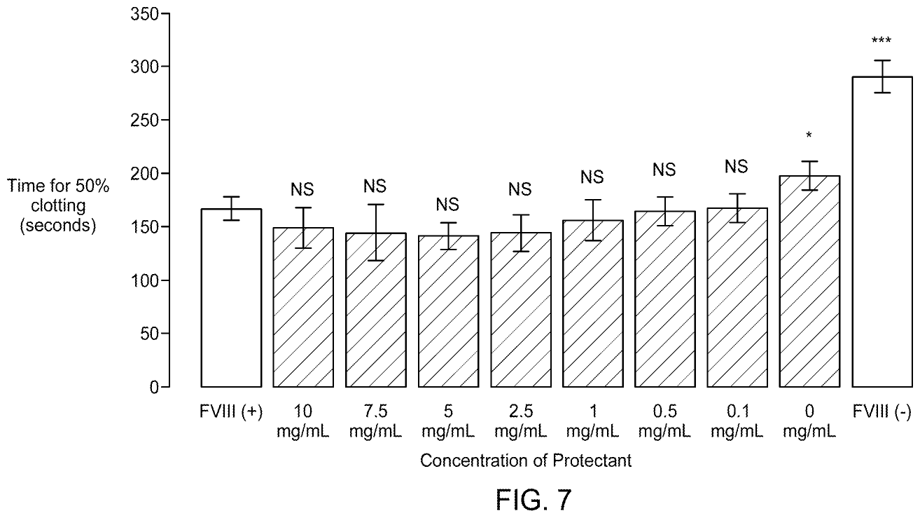

After observing the minimal protective capacity displayed by lysozyme, testing was performed on FVIII mixed with CAHS D or CAHS D engineered constructs prior to drying cycles. Results for the protective capacity of natural and engineered CAHS D constructs during repeated desiccation cycles are shown in FIG. 4 (CAHS D), FIG. 5 (CAHS D 2× Linker), FIG. 6 (CAHS D Full Length Proline), and FIG. 7 (CAHS D Linker Region). Specifically, these figures show histogram data of the 50% clotting time of FVIII after various treatments with CAHS D or an engineered construct subjected to repeated desiccation cycles.

In FIGS. 4-7, the FVIII(+) bar represents FVIII deficient human blood plasma treated with FVIII. “X mg/mL” notations beneath sample bars correspond to the concentrations of protein mixed with FVIII before desiccation. All notations above sample bars represent statistical comparison with FVIII(+). The FVIII(−) bar represents FVIII deficient human blood plasma treated with no FVIII. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***.

Table 2 shows gelation data, as a function of concentration, for example IDPs investigated herein CAHS D, CAHS D engineered constructs, AavLEA1, and Hero9-following the Example 6× Desiccation Protocol described above. As further described below, certain IDPs underwent a concentration dependent phase transition to form a hydrogel.

TABLE 2

| | Concentration of IDP at which the |
|---|---|
| IDP | sample undergoes gelation, mg/mL |
| CAHS D | 10 mg/mL |
| CAHS D 2× Linker | 5 mg/mL |
| CAHS D Full Length | No gelation at all concentrations investigated |
| Proline | |
| CAHS D Linker Region | No gelation at all concentrations investigated |
| AavLEA1 | No gelation at all concentrations investigated |
| Hero9 | No gelation at all concentrations investigated |

Referring to FIG. 4, at all concentrations tested, CAHS D provided full protection of FVIII, with low concentrations (0.1-0.5 mg/mL) providing accelerated clotting. While not wishing to be bound by any theory, it is believed that at low concentrations CAHS D causes crowding induced acceleration of clotting, possibly through a mechanism similar to that of trehalose. As shown in FIG. 4, while CAHS D at low concentrations accelerates clotting, this accelerating effect was observed to dissipate at higher concentrations.

CAHS D underwent a concentration dependent phase transition from a solution into a solid gel state. At concentrations of about 0.1 mg/mL to about 7.5 mg/mL CAHS D, samples remained fluid. At about 10 mg/mL and higher, CAHS D forms a hydrogel with samples taking on viscoelastic properties. While not wishing to be bound by any theory, it is believed that this counterintuitive result-whereby low concentrations of CAHS D accelerate clotting, but high concentrations do not-might be linked to CAHS D's propensity to form a hydrogel. The tendency of CAHS D to undergo a liquid to solid phase transition to form a hydrogel can be a result of having an amino terminus (N-term), a linker region, and a carboxy terminus (C-term). The formation of this hydrogel may impede the ability of CAHS D to confer protection to FVIII at higher concentrations

To investigate the role of CAHS D hydrogel formation in FVIII protection, the protection efficacy of engineered CAHS D variants (the engineered constructs) with different hydrogel formation behaviors.

An engineered construct of CAHS D, termed “2× Linker”, was first investigated. The 2× Linker protein includes the N- and C-termini of CAHS D held apart by a tandemly duplicated linker region and was determined to have an enhanced propensity for gelation. This 2× Linker variant forms a gel at a concentration of about 5 mg/mL, indicating that the 2× Linker variant forms a gel at concentrations lower than CAHS D (5 mg/mL for the 2× Linker vs. 10 mg/mL for the wild type CAHS D). Under the conditions tested, the results shown in FIG. 5 indicated that the 2× Linker engineered construct had a similar protective capacity to CAHS D with protection apparent at some lower concentrations before gelation had occurred. Consistent with the belief that faster gelation may decrease protective capacity during repeated desiccation cycles, the 2× Linker's protective capacity differed from CAHS D's in that the 2× Linker only provided protection at low to mid concentrations of 0.1, 0.5, and 5 mg/mL, as compared to CAHS D which conferred protection at or above 0.1 mg/mL (FIGS. 4 and 5).

A second engineered construct of CAHS D, termed “Full Length Proline” (or “FL Proline”) was investigated. This engineered construct has the entire length of the CAHS D protein, but has three prolines inserted into its C-terminal tail, making it unable to form a hydrogel. This is confirmed by the fact that the FL Proline engineered construct exhibited a lack of gelation of the FL Proline engineered construct at all concentrations investigated from about 0.1 mg/mL to about 20 mg/mL. FIG. 6 shows results for the protective capacity of the FL Proline engineered construct. For this non-gelling variant, protection at much higher concentrations relative to CAHS D was observed under the conditions tested. The results may indicate that by disrupting gelation, the FL Proline engineered construct can provide protection to FVIII at a wider range of concentrations.

Another engineered CAHS D construct, termed “Linker Region”, was investigated. The Linker Region variant comprises the internal linker region of CAHS D, which is unable to polymerize and form a gel. The Linker Region variant was constructed by removing both the N- and C-termini of CAHS D, which are required for hydrogel formation, resulting in just the linker region being present. The Linker Region engineered construct does not form a hydrogel at all concentrations tested from about 0.1 mg/mL to about 20 mg/mL. These concentrations are significantly above those concentrations that the wild type CAHS D would gel.

As shown in FIG. 7, when treated with any concentration of the Linker Region engineered construct at or above 1 mg/mL, robust and complete protection of FVIII function after desiccation was observed and this preservation of clotting speed was not seen to dissipate as concentrations of the Linker Region engineered construct increased. Under the conditions tested, the Linker Region engineered construct displayed the greatest range of protective concentrations, starting at about 0.1 mg/ml and extending to about 10 mg/mL. 20 mg/mL of the Linker Region engineered construct was also tested (data not shown), and provided protection of FVIII function after desiccation.

In contrast to CAHS D, alterations to the native sequence and conformational ensemble can influence CAHS D hydrogel formation, with expansion of the Linker Region leading to hydrogel formation at lower concentrations, or conversely, disruption of the N-term, linker, or C-term leading to no gel formation (Table 2). The data observed for CAHS D and the three CAHS D variants demonstrated that CAHS D and the engineered constructs can protect FVIII from desiccation. The data also indicates that protection may be effective within a concentration range where the CAHS D or variant thereof does not form a hydrogel. The results also indicate that engineered protein variants of CAHS D that do not gel (or do not substantially gel) can increase and extend the protective effect.

Overall, these results suggest that a desiccation-related IDP, CAHS D, provides increased protection to FVIII subjected to repeated desiccation cycles, relative to a control protein (lysozyme). Furthermore, by engineering CAHS D such that it can or cannot form a hydrogel, embodiments described herein can enhance or perturb the ability of CAHS D to confer protection to FVIII during repeated desiccation cycles.

### Example 2.3: AavLEA1 and Hero9 for Stabilizing FVIII During Repeated Cycles of Drying and Rehydration

Other desiccation-related IDPs beyond CAHS D and its variants were investigated to determine whether they can protect FVIII during repeated desiccation cycles. AavLEA1 is a Late Embryogenesis Abundant (LEA) protein from the desiccation tolerant nematode Aphelenchus avenae. Hero9 belongs to a newly discovered class of proteins called heat-resistant obscure (Hero) proteins, present in the human proteome, which despite being found in non-desiccation tolerant organisms, have been observed to confer protection against protein instability and aggregation. AavLEA1 and Hero9 did not undergo gelation at all concentrations tested from about 0.1 mg/mL to about 20 mg/mL.

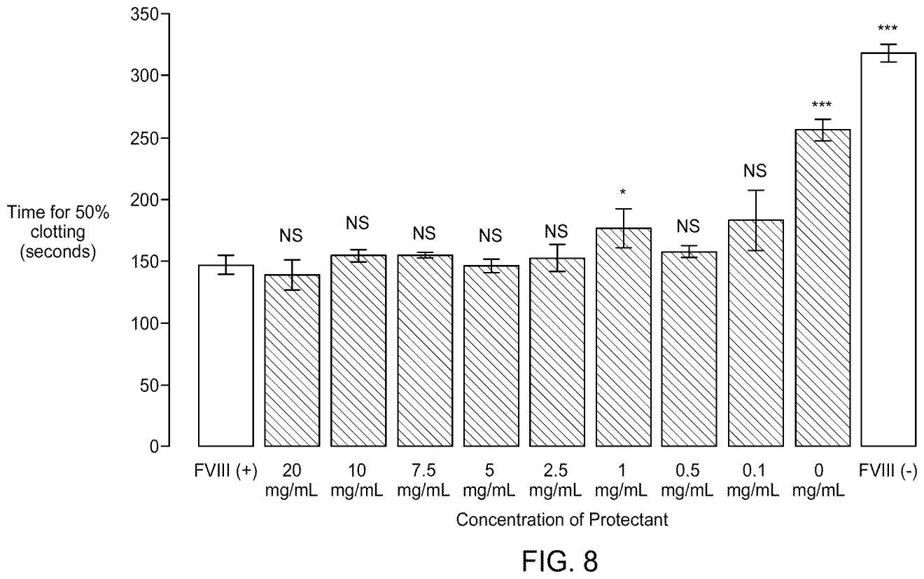

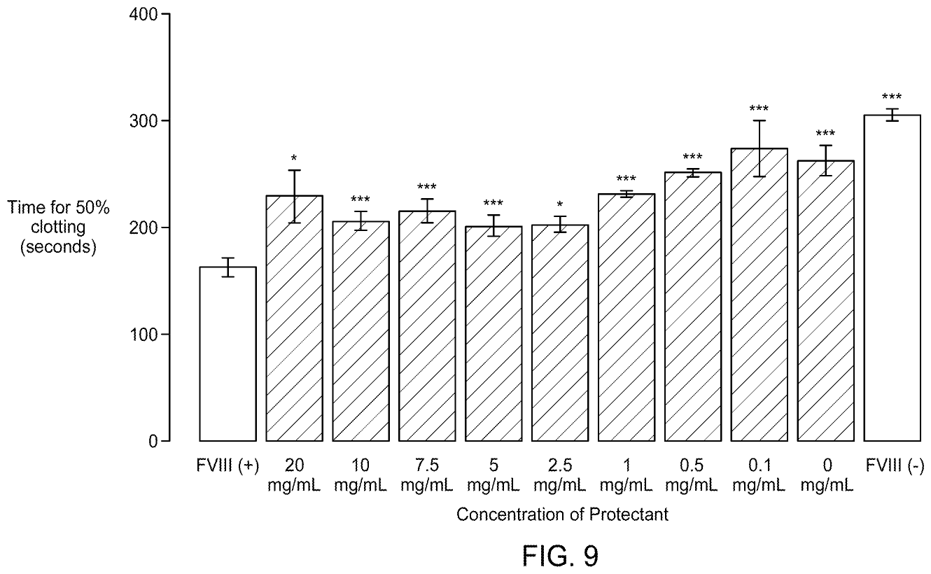

Results for non-CAHS IDPs' protective capacity during repeated desiccation cycles are shown in FIG. 8 (AavLea1) and FIG. 9 (Hero9). Specifically, these figures show histogram data of the 50% clotting time of FVIII after various treatments with AavLEA1 (FIG. 8) or Hero9 (FIG. 9) subjected to repeated desiccation cycles. The FVIII(+) bar represents FVIII deficient human blood plasma treated with FVIII. “X mg/mL” notations beneath sample bars correspond to the concentrations of AavLEA1 or Hero9, mixed with FVIII before desiccation. All notations above sample bars represent statistical comparison with FVIII(+). The FVIII(−) bar represents FVIII deficient human blood plasma treated with no FVIII. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***

Neither AavLEA1 nor Hero9 form hydrogels, and neither AavLEA1 nor Hero9 independently interfere with FVIII DHBP clotting. As shown in FIG. 8, AavLEA1 was observed to be fully or partially protective of FVIII after repeated desiccation at or above 0.1 mg/mL. This is in line with the measurements of CAHS D and Linker Region protection. This result further confirmed that desiccation related disordered proteins (CAHS and LEA proteins) that do not form hydrogels can provide increased protection to desiccated FVIII. Furthermore, the results show that these protein-based protectants can confer protection more effectively and at a wider range of concentrations as sugar-based protectants sucrose and trehalose.

As shown in FIG. 9, when Hero9 was mixed with FVIII prior to repeated desiccation cycles, it was observed to confer partial protection to FVIII at intermediate concentrations (about 2.5 mg/mL to about 10 mg/mL), suggesting that while Hero9 may be protective to some biomolecules under certain conditions. This effect may be limited during repeated drying cycles with FVIII.

Taken together, the results suggest that FVIII can be efficiently protected during repeated dehydration/rehydration cycles by both sugar and protein-based mediators of desiccation tolerance, but that robust preservation of FVIII by IDPs is not uniform and there may be sequence features as well as biochemical, and/or biophysical properties that make an IDP more or less protective for a particular client (the biologic) under a specific stress.

Overall, the results demonstrate that dry preservation of blood clotting factors—such as human blood clotting factor VIII—can be achieved using both sugar and protein-based excipients such as IDPs. Furthermore, the engineering of IDPs, such as CAHS D, can allow for the control of biochemical, biophysical, and material properties that influence the protective capacity of these IDPs.

### Example 2.4: Sugar-Based Mediators for Stabilizing FVIII During Heat Stress in a Dry State

Beyond repeated rehydration/dehydration cycles, another stress potentially encountered by a biologic during storage or transportation outside of the cold-chain is thermal stress. After establishing the protective capacity of sucrose and trehalose under repeated desiccation stress, the ability of these sugars' to stabilize FVIII in a dry state under heating stress was investigated.

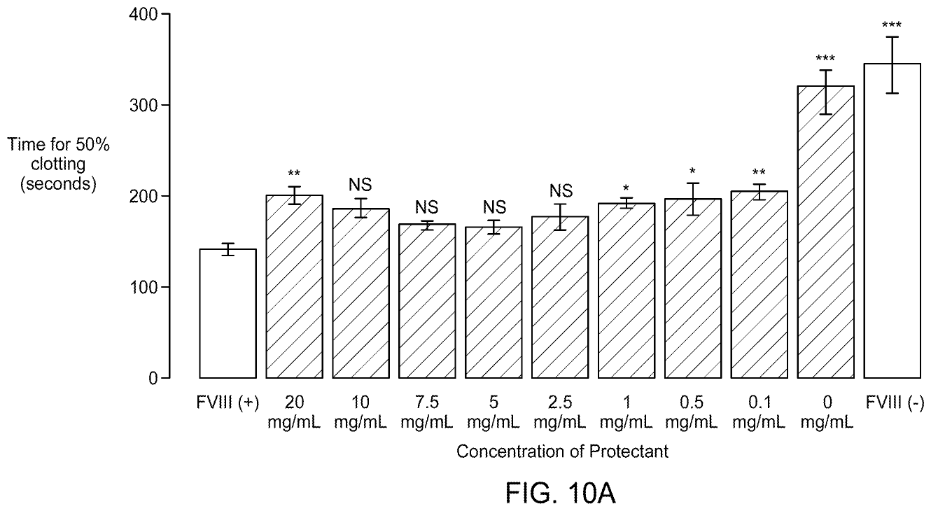

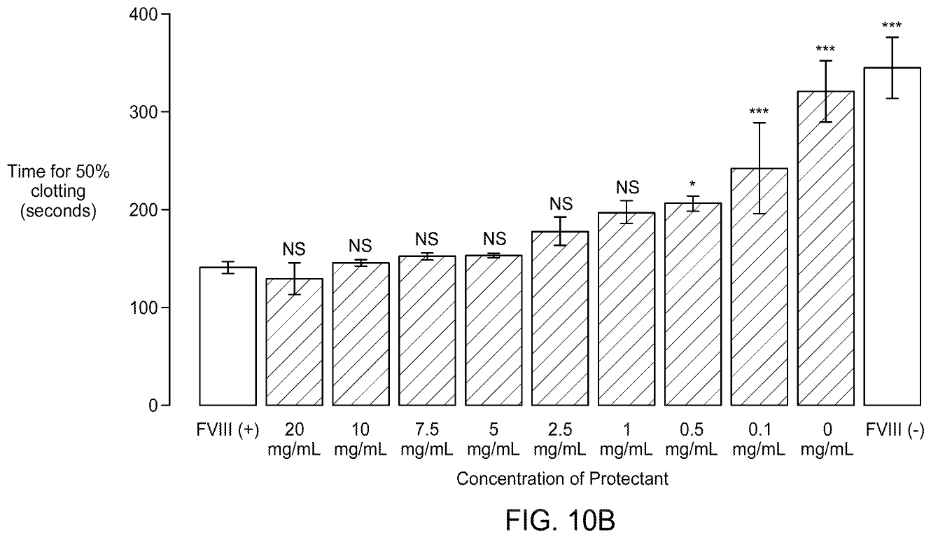

To test this, FVIII, which had been dried once, was subjected to about 48 hours of heating at about 95° C. FIGS. 10A and 10B show results of the non-reducing disaccharides, sucrose and trehalose, respectively, for stabilization of FVIII under thermal stress. Specifically, the data is histograms of the 50% clotting time of FVIII co-incubated with sucrose or trehalose. Specifically, these figures show histogram data of the 50% clotting time of FVIII co-incubated with sucrose (FIG. 10A) or Hero9 (FIG. 10B) subjected to repeated desiccation cycles.

In FIGS. 10A and 10B, the FVIII(+) bar represents FVIII deficient human blood plasma treated with unstressed FVIII. “X mg/mL” notations beneath sample bars correspond to the concentrations of non-reducing disaccharide mixed with FVIII before desiccation. All notations above sample bars represent statistical comparison with FVIII(+). The FVIII(−) bar represents FVIII deficient human blood plasma without FVIII supplementation. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***.

Heating of dry FVIII without trehalose or sucrose resulted in total loss of the factor's clotting ability as shown in FIGS. 10A and 10B, respectively. However, similar to 6× desiccation trials, sucrose and trehalose both conferred significant protection to FVIII under thermal stress. During heat stress in a dry state, sucrose provided partial or complete protection to FVIII at all concentrations tested from about 0.1 mg/mL to about 20 mg/mL as shown in FIG. 10A. During heat stress in a dry state, trehalose provided partial protection at low concentrations (from about 0.1 mg/mL to about 0.5 mg/mL) and full protection at higher concentrations (from about 1 mg/mL to about 20 mg/mL) as shown in FIG. 10B. These results demonstrate that sugar-based protectants can thermally protect the biologic FVIII.

### Example 2.5: CAHS D and Engineered CAHS D Variants for Stabilizing FVIII During Heat Stress in a Dry State

CAHS proteins, including CAHS D, are heat soluble in solution and have been shown to increase thermal tolerance when heterologously expressed in dry yeast, but their ability to confer long-term thermal tolerance to a client has yet to be established in vitro. Establishing the ability of dry-storage mediators to protect client biologics at elevated temperatures can be important since because most current storage methods that allow for cold-chain independent maintenance of FVIII at room temperature only work up to about 30° C. Since ambient air temperatures in many regions of the world exceed 30° C., identifying dry-storage mediators capable of stabilizing FVIII and other biologics beyond room temperature conditions can enable an alternative means of providing life-saving medicines to people everywhere.

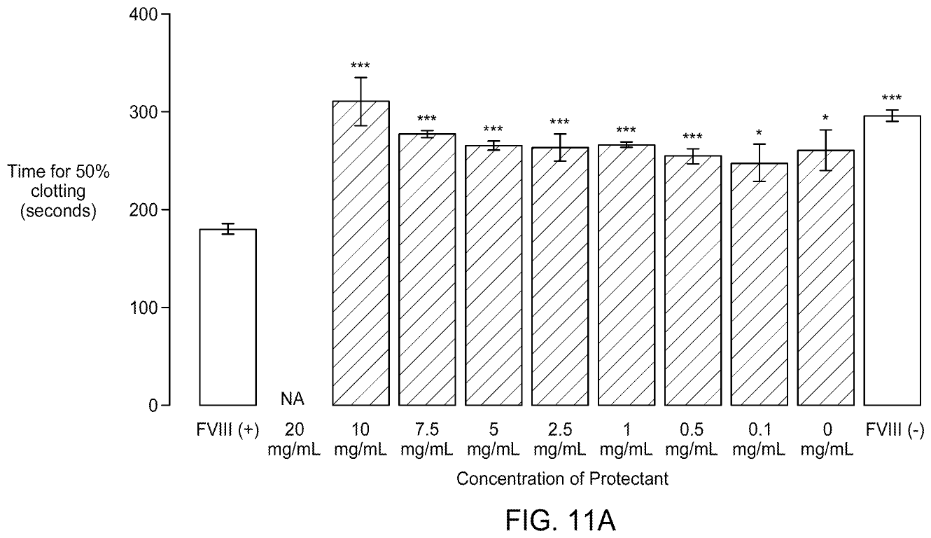

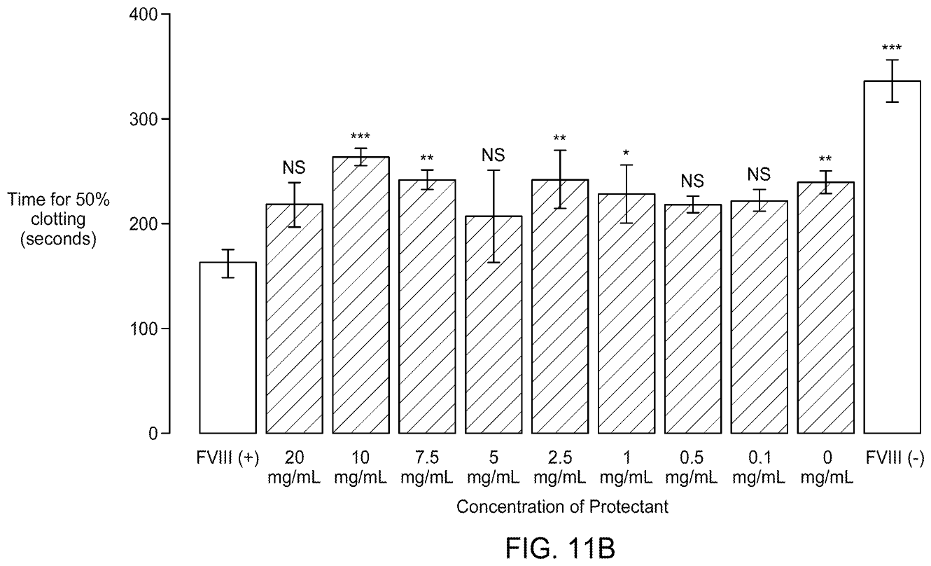

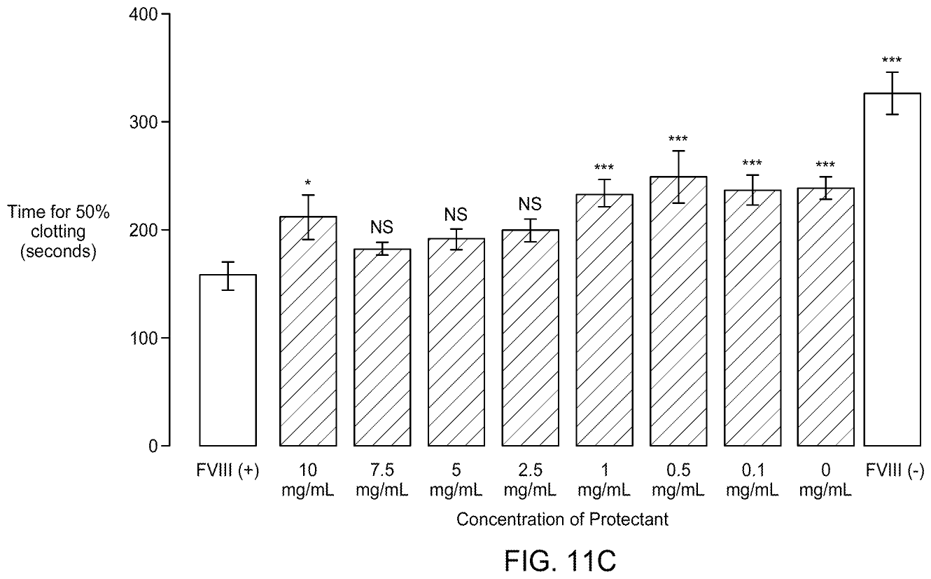

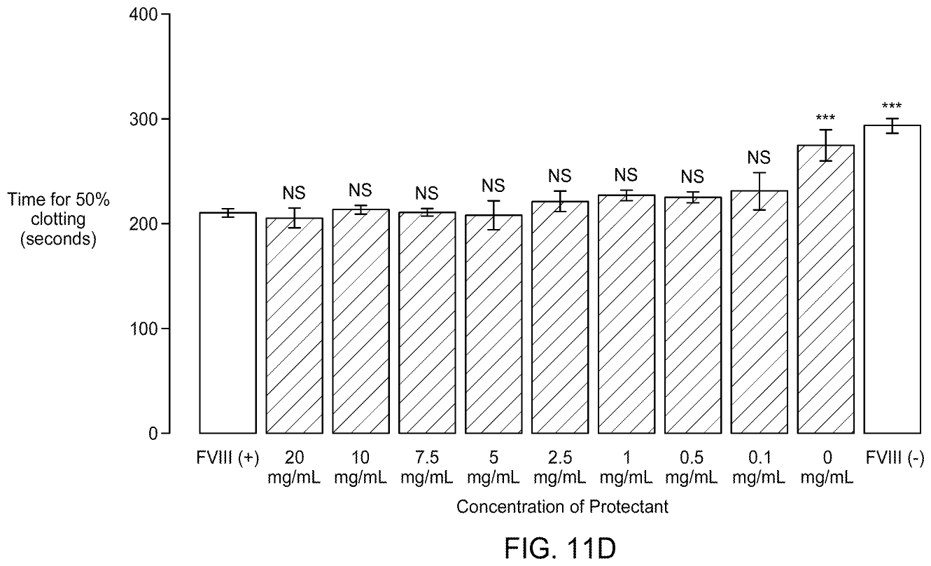

FIGS. 11A-11D show results for the use of CAHS D and engineered CAHS D constructs as stabilizers of FVIII under thermal stress in a dry state. Specifically, the data is histograms of 50% clotting time of FVIII treated with lysozyme (FIG. 11A), CAHS D (FIG. 11B), CAHS D Linker Region (FIG. 11A), or CAHS D 2× Linker (FIG. 11D) prior to desiccation and thermal stress. In FIGS. 11A-11D, the FVIII(+) bar represents FVIII deficient human blood plasma treated with non-perturbed FVIII. “X mg/mL” notation beneath sample bars correspond to the concentration of protein mixed with FVIII before desiccation. All notations above sample bars represent statistical comparison with FVIII(+). The FVIII(−) bar represents FVIII deficient human blood plasma not treated with FVIII. “NA” denotes that, although the relevant experiment was conducted, no clotting was observed during the entire duration of the clotting assay. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***.

As a negative control, lysozyme's ability to stabilize FVIII in a dry state under thermal stress (95° C. for 48 hours) was tested. As shown in FIG. 11A, lysozyme was not protective to FVIII under heating stress at any concentration. Furthermore, high concentrations (about 20 mg/mL) of lysozyme mixed with FVIII inhibited blood clotting all together. This complete inhibition was not observed for lysozyme in experiments conducted with repeated desiccation cycles (described above for lysozyme, but data not shown), suggesting that heating may impart some detrimental change to heat-insoluble lysozyme which interferes with plasma clotting.

As shown in FIG. 11B, co-incubation of FVIII with heat soluble CAHS D during heat stress showed no protection at any concentration. Similar to the 6× desiccation experiments, and as shown in FIG. 11C, the Linker Region engineered variant showed improved protection relative to CAHS D, with full or partial protection after heat stress observed at high concentrations of about 5 mg/mL to about 7.5 mg/mL. As shown in FIG. 11D, the CAHS D 2× Linker engineered construct was tested under thermal stress and robust protection of FVIII at all concentrations from 0.1 mg/mL to about 20 mg/L was observed.

These results suggested that sequence features as well as biophysical and biochemical properties can be changed to make IDP-based protectants more or less effective at preventing damage from thermal stress in the dry state. Specifically, gelation appears to not benefit, and can or even inhibit protection during repeated desiccation cycles, while gel formation appears to enhance thermal protection in the dry state.

### Example 2.6: AavLEA1 and Hero9 for Stabilizing FVIII During Heat Stress in a Dry State

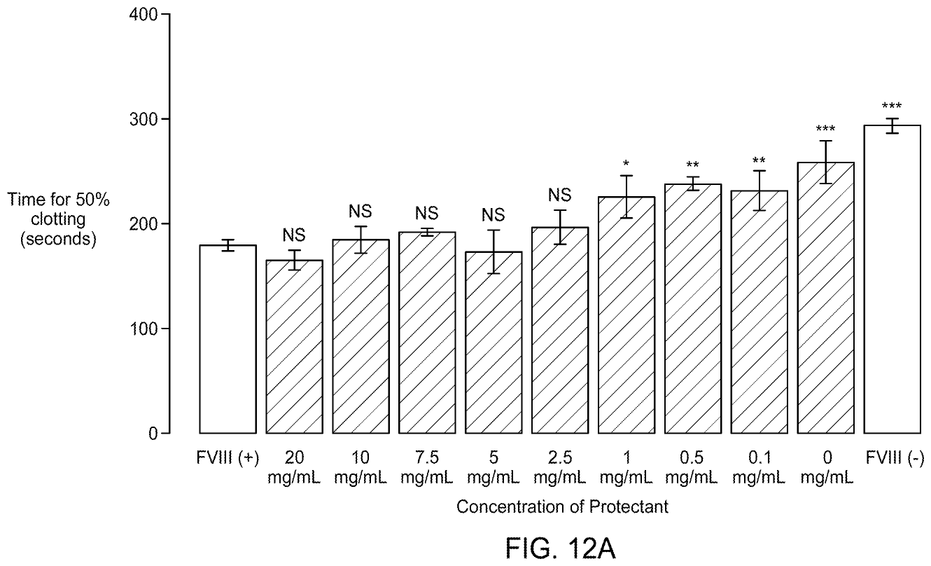

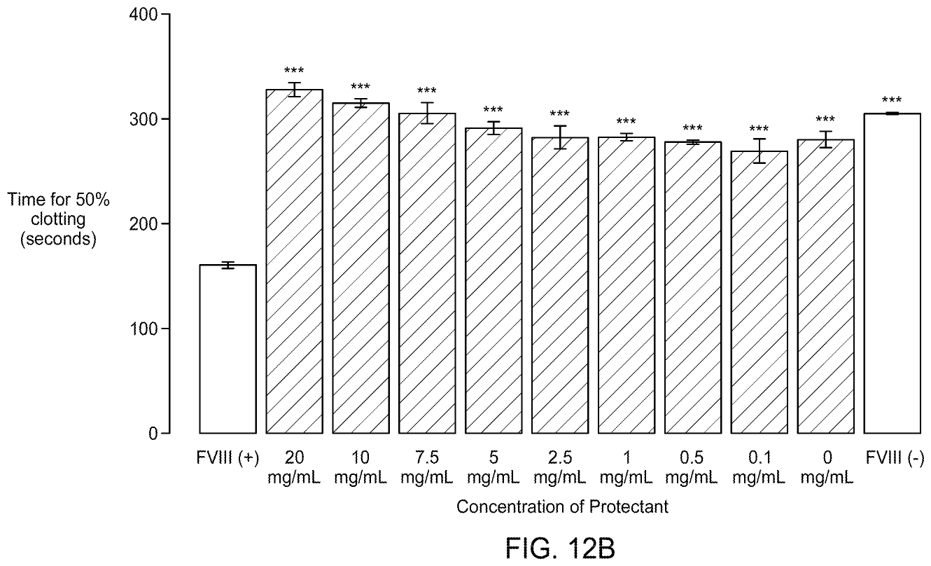

FVIII was incubated with AavLEA1 or Hero9 prior to desiccation and heating for 48 h at 95° C. FIGS. 12A and 12B show results AavLEA1 or Hero9 as stabilizers of FVIII under thermal stress in a dry state. Specifically, the data is histograms of 50% clotting time of FVIII treated with AavLEA1 (FIG. 12A) or Hero9 (FIG. 12B) prior to desiccation and thermal stress.

The FVIII(+) bar represents FVIII deficient human blood plasma treated with unperturbed FVIII. “X mg/mL” notations beneath sample bars correspond to the concentrations of AavLEA1 or Hero9 mixed with FVIII before desiccation. All notations above sample bars represent statistical comparison with FVIII(+). The FVIII(−) bar represents FVIII deficient human blood plasma treated with no FVIII. Notations above sample bars represent statistical significance determined by one-way ANOVA and Tukey's post-hoc test. Error bars represent bi-directional standard deviation. P value>0.05=NS, P<0.05=*, P<0.01=**, P<0.001=***.

As shown in FIG. 12A, after mixing AavLEA1 with FVIII and subjecting dry samples to thermal stress, FVIII function was observed to have no protection under heat stress at low to medium concentrations (from about 0.1 mg/mL to about 1 mg/mL) but completely preserved FVIII function at middle to high concentrations (from about 2.5 mg/mL to about 20 mg/mL).

As shown in FIG. 12B, Hero9 did not confer protection to FVIII after heating at any concentrations, and became antagonistic, causing slowed 50% clotting time, at high concentrations (from about 7.5 mg/mL to about 20 mg/mL). Taken together, the data indicated that while other desiccation-related and heat soluble IDPs can protect FVIII against thermal stress, this is not a ubiquitous feature of all heat soluble IDPs.

### Example 2.7: Time Course Investigations

Considering the practical application of using dry preservation, or xeroprotection, as a means of stabilization of labile biologics during their transport and storage, investigations into shelf-stable FVIII in a dry state were performed. The Linker Region engineered construct was investigated for this study. It was determined that the CAHS D Linker Region engineered construct can stabilize FVIII in a dry state for at least 10 weeks.

FVIII was incubated with 10 mg/mL of Linker Region engineered construct or without the Linker Region engineered construct. Samples were split, with one sample being left hydrated while the other was dried. Triplicate samples were prepared and examined every week for ten weeks. FIGS. 13A and 13B show results from the CAHS D Linker Region time course stabilization. Specifically, the data is for 50% clotting time of FVIII in a hydrated or dry state with or without addition of 10 mg/mL of CAHS D Linker Region. Samples were prepared and left dry/hydrated for 1 to 10 weeks before testing in our clotting assay. Data for each weekly time point when a 50% clotting time could be established is shown in FIG. 13A. FIG. 13B shows a statistical comparison between the first and last obtainable 50% clotting time. Notations above bar represent statistical comparisons between the first and the last time point taken using a 2-tailed T-test. Error bars represent bi-directional standard deviation. P value>0.05=NS, P<0.05=*, P<0.01=**, P<0.001=***.

As shown in FIGS. 13A and 13B, FVIII left in a hydrated state with or without the Linker Region engineered construct lost a significant amount of functionality over the time course (p<0.01). Dried samples without the Linker Region engineered construct lost a significant amount of clotting ability over the course of ten weeks (p<0.001). Samples of FVIII dried with 10 mg/mL of the Linker Region engineered construct did not statistically change over the course of 10 weeks. Overall, the data indicated that, while FVIII clotting potential decreases over time when stored under hydrated conditions (with or without the Linker Region engineered construct) or when stored dry without an excipient, the addition of the Linker Region engineered construct at a concentration of about 10 mg/mL can be sufficient to stabilize FVIII function for at least 10 weeks in a dry state.

### 3. Non-Limiting Results and Discussion

While conventional wisdom dictates that FVIII be kept using cold-storage, state-of-the-art technologies such as PEGylation and other excipient practices have resulted in FVIII products that can be stored at room temperature. However, medical professionals still identify problems with FVIII storage as the room-temperature storage range is small, usually only allowing storage at temperatures up to 30° C., and even in developed parts of the world this poses issues for at-home storage and treatment.

To overcome this challenge, embodiments of the present disclosure can enable storage at room temperature and greater (up to, for example, about 95° C.) using an IDP, a sugar, or combinations thereof. For example, sugars can provide protection to FVIII during repeated desiccation cycles in a concentration dependent fashion. As another example of the present disclosure, CAHS D provides protection to FVIII during repeated desiccation cycles at all concentrations tested. Both gelling and non-gelling engineered constructs of CAHS D proteins described herein can be utilized to protect FVIII, with the non-gelling variant protecting FVIII at a wider range of concentrations. This protection can increase in a concentration dependent fashion. In addition, the protective effect of AavLEA1, a protective protein derived from a nematode worm, which does not gel, can provide robust protection to FVIII across a wide range of concentrations during repeated desiccation cycles.

Similar to repeated desiccation cycles, an IDP, a sugar, or combinations thereof can be utilized for protecting FVIII during thermal stress in a dry state. For example, it was found that the strong gelation capacity of the 2× Linker variant, while inhibitory to protection during repeated desiccation cycles, can be protective under thermal stress, suggesting that different engineered biophysical and material properties can change a protein's protective capacity for FVIII under different storage/stress conditions.

Embodiments described herein can also be utilized to extend the shelf life of dry FVIII. For example, the CAHS D Linker Region engineered construct can be utilized to extend the shelf-life of dry FVIII. Under dry conditions, the stability of FVIII co-incubated with the Linker Region variant was unchanged over 10 weeks, while FVIII dried without excipients (for example, a sugar and/or an IDP) degraded significantly over that period of time, as did hydrated samples.

Overall, embodiments described herein can be utilized to stabilize biologics, such as Human Blood Clotting Factor FVIII, during repeated desiccation cycles and under thermal stress. That is, and in some examples, natural and/or engineered mediators such as a sugar, an IDP, or combinations thereof can protect biologics in non-cold-storage regimes. The inventors show that, for example, protein engineering can be used to change the protective capacity of natural mediators of desiccation tolerance to enhance them for different storage conditions such as ambient or even severely elevated temperatures. In contrast, conventional technologies and stabilization methods such as PEGylation are unable to stabilize dry biologics, such as dry FVIII, under such conditions.

Embodiments described herein can be utilized for the dry preservation of biologics, such as Human Blood Clotting Factor VIII (FVIII), a key molecule in the intrinsic blood clotting pathway with numerous clinical applications. For example, sugars and/or IDPs can be utilized to preserve biologics in a dry state and even under thermal stress.

While FVIII can be stabilized under repeated desiccation cycles and thermal stress, the mediators that do best at preventing damage during these distinct stresses can be different under the conditions investigated. The non-gelling AavLEA1 protein and CAHS Linker Region variant prevented damage to FVIII in repeated desiccation cycles the CAHS Linker Region variant), while the gelling variant CAHS D 2× Linker outperformed all other excipients in preserving FVIII function under thermal stress. The functional ramifications of the phase transition to hydrogel as it relates to desiccation tolerance has not previously been empirically tested. The results described herein demonstrate that the phase of an IDP can influence its protective capacity, and that stress-related IDPs can be engineered to serve specific functions with regard to biologic stabilization.

In addition, the use of IDPs can augment functions and protective mechanisms using different chemical environments. For example, the inventors have also found that IDPs described herein can work synergistically with sugars (such as trehalose, among others) to promote desiccation tolerance both in vitro and in vivo. This suggests that not only can IDP protective function be altered by changes to their sequence, but also by modulation of their chemical environment. As such, the combination of CAHS engineered proteins and sugars could elicit synergistic effects in stabilizing FVIII and other biologics under different dry storage conditions.

Embodiments described herein show that dry preservation methods can be effective in protecting biologics, offering a convenient, logistically simple, and economically viable means of stabilizing life-saving medicines. This can be beneficial for global health initiatives in remote or developing parts of the world, and for fostering a safe and productive space economy which will be reliant on new technologies that break our dependence on the cold-chain for the storage of medicine, food, and other biomolecules.

### EMBODIMENTS LISTING

The present disclosure provides, among others, the following aspects, each of which can be considered as optionally including any alternate embodiments:
Clause A1. An intrinsically disordered protein for stabilizing a blood clotting factor protein, comprising:
an amino acid sequence having at least 80% identity to one or more of SEQ ID NOs: 2, 4, 6, 8, 10, 12, or a complement thereof.
Clause A2. A nucleic acid comprising a nucleotide sequence encoding the intrinsically disordered protein of Clause A1.
Clause A3. The intrinsically disordered protein of Clause A1 or Clause A2, wherein the blood clotting factor protein comprises human blood clotting factor VIII.
Clause B1. A method for stabilizing a blood clotting factor protein, the method comprising:
introducing an intrinsically disordered protein with a blood clotting factor protein to form a composition, the composition characterized as stabilizing the blood clotting factor protein of the composition in a dry state at a temperature of ambient temperature or higher.
Clause B2. The method of Clause B14, wherein the composition further comprises a disaccharide.
Clause B3. The method of Clause B2, wherein the disaccharide comprises trehalose, sucrose, or combinations thereof.
Clause B4. The method of any one of Clauses B1-B3, wherein the intrinsically disordered protein comprises a cytoplasmic abundant heat soluble (CAHS) protein, a modified CAHS protein, a late embryogenesis abundant (LEA) protein, a modified LEA protein, a heat-resistant obscure (Hero) protein, a modified Hero protein, or combinations thereof.
Clause B5. The method of any one of Clauses B1-B4, wherein the composition is characterized as stabilizing the blood clotting factor protein of the composition in a dry state at a temperature of up to about 95° C.
Clause B6. The method of any one of Clauses B1-B5, wherein the composition comprises 15 wt % or less of water based on a total wt % of the composition, the total wt % of the composition not to exceed 100 wt %.
Clause B7. The method of any one of Clauses B1-B6, wherein the intrinsically disordered protein comprises an amino acid sequence having at least 80% identity to one or more of SEQ ID NOs: 2, 4, 6, 8, 10, 12, or a complement thereof.
Clause B8. The method of any one of Clauses B1-B7, wherein the intrinsically disordered protein comprises an amino acid sequence encoded by a nucleotide sequence having at least about 80% identity to one or more of SEQ ID NOs: 1, 3, 5, 7, 9, 11, or a complement thereof.
Clause C1. A composition, comprising:
an intrinsically disordered protein;
a blood clotting factor protein in a dry state; and
15 wt % or less of water based on a total wt % of the composition, the total wt % of the composition not to exceed 100 wt %, the composition being characterized as stabilizing the blood clotting factor protein of the composition at a temperature of ambient temperature or higher.
Clause C2. The composition of Clause C1, further comprising a disaccharide.
Clause C3. The composition of Clause C1 or Clause C2, wherein the intrinsically disordered protein comprises a cytoplasmic abundant heat soluble (CAHS) protein, a modified CAHS protein, a late embryogenesis abundant (LEA) protein, a modified LEA protein, a heat-resistant obscure (Hero) protein, a modified Hero protein, or combinations thereof.
Clause C4. The composition of any one of Clauses C1-C3, wherein the composition is characterized as stabilizing the blood clotting factor protein of the composition in a dry state at a temperature of up to about 95° C.
Clause C5. The composition of any one of Clauses C1-C4, wherein the intrinsically disordered protein comprises an amino acid sequence having at least 50% identity to one or more of SEQ ID NOs: 2, 4, 6, 8, 10, 12, or a complement thereof.
Clause C6. The composition of any one of Clauses C1-C5, wherein the intrinsically disordered protein comprises an amino acid sequence having at least 80% identity to one or more of SEQ ID NOs: 2, 4, 6, 8, 10, 12, or a complement thereof.
Clause C7. The composition of any one of Clauses C1-C6, wherein the intrinsically disordered protein comprises an amino acid sequence encoded by a nucleotide sequence having at least about 50% identity to one or more of SEQ ID NOs: 1, 3, 5, 7, 9, 11, or a complement thereof.
Clause C8. A pharmaceutical composition, comprising:
the composition of any one of Clauses C1-C7; and
a pharmaceutically acceptable carrier, excipient, adjuvant, or combinations thereof.
Clause C9. The pharmaceutical composition of Clause C8, wherein the composition further comprises a disaccharide, the disaccharide different from the pharmaceutically acceptable carrier, excipient, and adjuvant.

Embodiments described herein generally relate to the stabilization of biologics, such as blood clotting factors, in a dry state. Unlike conventional technologies that utilize cold-chain refrigeration, freezing, lyophilization, PEGylation to store sensitive biologics, embodiments described herein can protect or preserve biologics in a dry state at non-ideal temperatures (e.g., about 8° C. or more, such as about ambient temperature, such as temperatures higher than ambient temperature). Embodiments described herein enable dry preservation methods that are effective in protecting biologics, thereby offering a convenient, logistically simple, and economically viable means of stabilizing life-saving medicines. This will be beneficial for global health initiatives in remote or developing parts of the world, but also in fostering a safe and productive space economy.

As is apparent from the foregoing general description and the specific embodiments, while forms of the embodiments have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa, for example, the terms “comprising,” “consisting essentially of,” “consisting of” also include the product of the combinations of elements listed after the term.

Furthermore, although embodiments described herein may achieve advantages over other possible solutions or over the prior art, whether or not a particular advantage is achieved by a given embodiment is not limiting of the scope of the present disclosure. Thus, the aspects, features, embodiments, and advantages described are merely illustrative and are not considered elements or limitations of the appended claims except where explicitly recited in a claim(s). Likewise, reference to “the disclosure” shall not be construed as a generalization of any inventive subject matter described herein and shall not be considered to be an element or limitation of the appended claims except where explicitly recited in a claim(s).

References cited herein are incorporated by reference herein in their entirety to indicate the state of the art as of their publication or filing date and it is intended that this information can be employed herein, if needed, to exclude specific embodiments that are in the prior art.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. The degree of sequence identity between two or more nucleotide sequences may be calculated using a known computer algorithm for sequence alignment such as NCBI BLAST, using standard settings. In determining the degree of sequence identity between two amino acid sequences, a skilled artisan can consider “conservative” amino acid substitutions, which can be described as amino acid substitutions in which an amino acid residue is replaced with another amino acid residue of similar chemical structure and which has little or essentially no influence on the function, activity, or other biological properties of the polypeptide.

Such conservative substitutions can be substitutions in which one amino acid within the following groups (a)-(e) is substituted by another amino acid residue within the same group: (a) small aliphatic, nonpolar or slightly polar residues: alanine (Ala), serine (Ser), threonine (Thr), proline (Pro), and glycine (Gly); (b) polar, negatively charged residues and their (uncharged) amides: aspartic acid (Asp), asparagine (Asn), glutamic acid (Glu), and glutamine (Gln); (c) polar, positively charged residues: histidine (His), arginine (Arg), and lycine (Lys); (d) large aliphatic, nonpolar residues: methionine (Met), leucine (Leu), isoleucine (Ile), valine (Val), and cysteine (Cys); and (e) aromatic residues: phenylalanine (Phe), tyrosine (Tyr), and tryptophan (Trp).

Particularly suitable conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln, or into Glu; Met into Leu, into Tyr, or into Ile; Phe into Met, into Leu, or into Tyr; Ser into Thr; Thr into Ser; Trp into Tyr; Tyr into Trp; and/or Phe into Val, into Ile, or into Leu.

Any suitable amino acid substitutions applied to polypeptides described herein can also be based on the analysis of the frequencies of amino acid variations between homologous proteins of G. E. Schulz and R. H. Schirmer, Principles of Protein Structure, Springer, New York, 1978, on the analyses of structure forming potentials developed by P. Y. Chou and G. D. Fasman, Biochemistry, 1974, 13, 211, and Adv. Enzymol., A. Meister ed., J. Wiley and Sons, 1978, 47, 45-149 and on the analysis of hydrophobicity patterns in proteins developed by D. Eisenberg et al., Proc. Nat. Acad. Sci. USA, 1984, 81, 140-144; J. Kyte and R. F. Doolittle, J. Mol. Biol., 1982, 157, 105-132, and D. M. Engelman et al., Annu. Rev. Biophys. Biophys. Chem., 1986, 15, 321-53, all of which are incorporated herein in their entirety by reference.

As used herein, the term “pharmaceutically acceptable” means that the carrier, excipient, or adjuvant is compatible with the other ingredients of the composition and not substantially deleterious to the recipient thereof and/or that such carrier or adjuvant is approved or approvable for inclusion in a pharmaceutical composition for parenteral administration to humans.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, embodiments comprising “an IDP” include embodiments comprising one, two, or more IDPs, or at least one IDP, unless specified to the contrary or the context clearly indicates only one IDP is included.

While the foregoing is directed to embodiments of the present disclosure, other and further aspects of the disclosure may be devised without departing from the basic scope thereof, and the scope thereof is determined by the claims that follow.

What is claimed is:

1. An intrinsically disordered protein for stabilizing a blood clotting factor protein, comprising:
an amino acid sequence having at least 80% identity to one or more of SEQ ID NOs: 2, 4, 6, 8, 10, 12, or a complement thereof.

2. A nucleic acid comprising a nucleotide sequence encoding the intrinsically disordered protein of claim 1.

3. The intrinsically disordered protein of claim 1, wherein the blood clotting factor protein comprises human blood clotting factor VIII.

4. A method, comprising:
introducing an intrinsically disordered protein with a blood clotting factor protein to form a composition, the composition characterized as stabilizing the blood clotting factor protein of the composition in a dry state at a temperature of ambient temperature or higher.

5. The method of claim 4, wherein the composition further comprises a disaccharide.

6. The method of claim 5, wherein the disaccharide comprises trehalose, sucrose, or combinations thereof.

7. The method of claim 4, wherein the intrinsically disordered protein comprises a cytoplasmic abundant heat soluble (CAHS) protein, a modified CAHS protein, a late embryogenesis abundant (LEA) protein, a modified LEA protein, a heat-resistant obscure (Hero) protein, a modified Hero protein, or combinations thereof.

8. The method of claim 4, wherein the composition is characterized as stabilizing the blood clotting factor protein of the composition in a dry state at a temperature of up to about 95° C.

9. The method of claim 4, wherein the composition comprises 15 wt % or less of water based on a total wt % of the composition, the total wt % of the composition not to exceed 100 wt %.

10. The method of claim 4, wherein the intrinsically disordered protein comprises an amino acid sequence having at least 80% identity to one or more of SEQ ID NOs: 2, 4, 6, 8, 10, 12, or a complement thereof.

11. The method of claim 4, wherein the intrinsically disordered protein comprises an amino acid sequence encoded by a nucleotide sequence having at least about 80% identity to one or more of SEQ ID NOs: 1, 3, 5, 7, 9, 11, or a complement thereof.

12. A composition, comprising:
an intrinsically disordered protein;
a blood clotting factor protein; and
15 wt % or less of water based on a total wt % of the composition, the total wt % of the composition not to exceed 100 wt %, the composition being characterized as stabilizing the blood clotting factor protein of the composition at a temperature of ambient temperature or higher.

13. The composition of claim 12, further comprising a disaccharide.

14. The composition of claim 12, wherein the intrinsically disordered protein comprises a cytoplasmic abundant heat soluble (CAHS) protein, a modified CAHS protein, a late embryogenesis abundant (LEA) protein, a modified LEA protein, a heat-resistant obscure (Hero) protein, a modified Hero protein, or combinations thereof.

15. The composition of claim 12, wherein the composition is characterized as stabilizing the blood clotting factor protein of the composition in a dry state at a temperature of up to about 95° C.

16. The composition of claim 12, wherein the intrinsically disordered protein comprises an amino acid sequence having at least 50% identity to one or more of SEQ ID NOs: 2, 4, 6, 8, 10, 12, or a complement thereof.

17. The composition of claim 12, wherein the intrinsically disordered protein comprises an amino acid sequence having at least 80% identity to one or more of SEQ ID NOs: 2, 4, 6, 8, 10, 12, or a complement thereof.

18. The composition of claim 12, wherein the intrinsically disordered protein comprises an amino acid sequence encoded by a nucleotide sequence having at least about 50% identity to one or more of SEQ ID NOs: 1, 3, 5, 7, 9, 11, or a complement thereof.

19. A pharmaceutical composition, comprising:
the composition of claim 12; and
a pharmaceutically acceptable carrier, excipient, adjuvant, or combinations thereof.

20. The pharmaceutical composition of claim 19, wherein the composition further comprises a disaccharide, the disaccharide different from the pharmaceutically acceptable carrier, excipient, and adjuvant.
